(19) United States
(12) Patent Application Publication
(10) Pub. No.: US 2014/0058189 A1
(43) Pub. Date: Feb. 27, 2014

(54) SYSTEMS AND METHODS USING BRAIN STIMULATION FOR TREATING DISORDERS

(71) Applicant: William F. Stubbeman, Santa Monica, CA (US)

(72) Inventor: William F. Stubbeman, Santa Monica, CA (US)

(21) Appl. No.: 13/768,819

(22) Filed: Feb. 15, 2013

Related U.S. Application Data
(60) Provisional application No. 61/601,005, filed on Feb. 20, 2012.

(51) Int. Cl. A61N 2/00; A61M 21/02

(52) U.S. Cl. CPC A61N 2/002; A61N 2/006; A61M 21/02
USPC 600/13

(57) ABSTRACT

Systems and methods are disclosed for electromagnetically stimulating a brain and pairing temporally associated sensory stimulation to treat a neurologic or psychiatric disorder or to enhance cognitive, motor, social, or psychological skills. In addition, a non-invasive brain stimulation device is configured to stimulate a patient's brain by emitting an electromagnetic field based on certain stimulation parameters that may be dynamically adjusted based on measurements regarding brain activity. An additional exemplary embodiment of the disclosed subject matter is a method of neuroplastic augmentation using brain stimulation designed to augment, hasten, enhance, optimize, or improve a secondary neurologic or psychiatric treatment for a brain illness.

### CROSS-REFERENCE TO RELATED APPLICATION

The present application claims benefit of and priority to U.S. Provisional Patent Application Ser. No. 61/601,005 filed on Feb. 20, 2012 entitled “Systems and Methods Using Brain Stimulation for Treating Disorders,” the entire contents of which are hereby incorporated by reference herein.

### BACKGROUND

The present disclosure generally relates to novel treatment systems and methods, and more specifically to systems and methods capable of treating neurologic or psychiatric disorders, as well as enhancing skill sets.

The brain is the most complex of all the organs in the human body. Perhaps due at least in part to such complexity, the brain also happens to be the organ with the highest prevalence of illness in the population. About 46% of those living in the United States will suffer from a diagnosable psychiatric disorder in their lifetime. Lifetime prevalence by illness categories are: 29% anxiety disorders, 21% mood disorders, 15% substance disorders, 15% personality disorders, 8% attention deficit hyperactivity disorders, 3% psychotic disorders, and 3% autism spectrum disorders (Kessler et al. 2005; Kim et al. 2011; Perala et al. 2007).

Neurologic disorders are also highly prevalent in the population. Tinnitus afflicts about 10% of the population (Shargorodsky et al. 2011). Chronic pain is reported by a third of the population and one in seven people suffer daily (Mantyselka et al. 2011; Reid et al. 2011). Chronic lower back pain, which accounts for approximately 3% of all physician office visits in the United States and hundreds of billions of dollars in annual treatment costs, is now also thought to have its origins in the brain (Wand et al. 2011).

Fortunately for the billions of people suffering from brain disorders, treatments are being developed that show a great deal of promise in treating such illnesses. Unfortunately, current treatments prescribed to patients suffering from psychiatric or neurologic disorders are merely palliative, at best. For example, treatments for neurologic disorders, such as stroke, epilepsy and dementia, with a lifetime prevalence of 6%, are often ineffective and do not address the root cause of the illness (MacDonald et al. 1999).

### I. Chemical Treatments

Treatments offered for people suffering from brain disorders generally fall into one of two categories: chemical (psychopharmacologic) or neurostimulation (brain or peripheral nerve stimulation). The majority of psychiatric and neurologic illnesses are treated chemically, i.e., with pharmacologic agents.

Neuropharmacologic and psychopharmacologic agents act at synaptic receptors to alter certain brain inputs in ways that reduce symptoms of mental and neurologic illness. However, chemical intervention has significant drawbacks. Often, the medication(s) must be taken for the rest of a patient's life to keep potentially disabling symptoms under control. If the medication regimen is stopped, the symptoms usually return, sometimes to a greater degree than were initially present, because the underlying pathologic wiring of the brain is not significantly altered. There are also potentially serious side effects, compliance problems, and widespread lack of efficacy (one-third of depressed and schizophrenic patients do not respond to known pharmacologic treatments) associated with medications.

### II. Neurostimulation Treatments

In contrast to chemical treatments, neurostimulation involves modulation of the nervous system by electrically activating neurons in the body through stimulation. Neurostimulation treatments may also be neuroplastic. Neuroplasticity is the ability of the brain to rewire itself permanently in response to changing external or internal stimuli. The brain has a high degree of neuroplasticity in childhood, enabling children to learn in a highly efficient manner and heal from potentially devastating neural injuries. However, neuroplastic properties of the brain diminish rapidly with age (Doidge et al. 2007). As such, neuroplastic constraints greatly limit the effectiveness of most medical therapy of psychiatric or neurologic illnesses to a slow, transient, or partial response.

Although relatively undeveloped, neurostimulation techniques have shown promise in treating nervous system illnesses, including those that are refractory to chemical treatment methods. Neurostimulation techniques generally fall into one of two categories: peripheral nerve stimulation or brain stimulation.

A. Peripheral Nerve Stimulation

Peripheral nerve stimulation activates nerves outside the brain. An example of peripheral nerve stimulation is vagus nerve stimulation (“VNS”). The vagus nerve is a peripheral nerve important for homeostatic physiologic regulation (e.g., decreases heart rate, activates digestive tract). VNS typically consists of surgically implanting an electronic stimulation device into the thoracic cavity and attaching linked electrodes to the left vagus nerve. Stimulation of the vagus nerve transmits electrical impulses upward through the chest, neck, and skull base into the brainstem.

VNS has been available since 1997 to treat intractable epilepsy and was approved by the FDA in 2005 for use in treatment-resistant depression. VNS techniques have also been reported to induce neuroplasticity in animals. (Engineer et al. 2011; United States Patent Applications 20100003656, 20100004705, 20100004717, 20110282404). However, VNS-triggered enhancement of brain neuroplasticity is not known to have been demonstrated in humans nor independently replicated in animals.

Even if VNS were eventually shown to effect neuroplasticity in humans, VNS has a number of significant drawbacks. Because VNS stimulates the laryngeal nerve, it is associated with many side effects. Two thirds of VNS patients experience changes in the sound of their voice, roughly half experience excessive coughing, and a third complain of throat inflammation and pain. (Hsieh et al. 2008). Cardiac arrhythmias and vocal chord spasm leading to upper airway obstruction have been reported (Hatton et al. 2006). Other side effects include headache, nausea, vomiting, stomach upset, shortness of breath, numbness, and tingling. (Sackeim et al. 2000).

In sum, VNS disadvantageously uses electrical stimulation (cannot pass through tissue unimpeded, as opposed to magnetic stimulation) and works through peripheral nerves (eliminating the possibility of tailoring the treatment to specific brain regions and causing severe side effects). VNS is also typically invasive (involving surgery to implant the vagal nerve stimulator), exposes the patient to the risks of general anesthesia, and entails the risk of infection (due to the surgical implantation of a foreign object in the body).

A related peripheral nerve stimulation technique that is non-invasive is transcutaneous vagal nerve stimulation (“t-VNS”), which stimulates the auricular branch of the vagus nerve. However, at least one study has found t-VNS to be ineffective in enhancing neuroplasticity. (Engineer et al. 2011).

B. Brain Stimulation

In contrast to peripheral nerve stimulation, brain stimulation directly stimulates the brain. Transcranial magnetic stimulation (“TMS”) is an example of brain stimulation. TMS is a non-invasive technique that typically involves placing a coil near the patient's head to depolarize or hyperpolarize neurons of the brain. In particular, TMS uses electromagnetic induction to induce weak electrical currents using a rapidly changing magnetic field to cause activity in specific or general brain regions.

TMS has diagnostic uses including determining the contribution of cortical networks to specific cognitive functions by disrupting activity in the focal brain region. TMS also has a number of therapeutic uses. For example, a variant of single pulse TMS is repetitive transcranial magnetic stimulation (“rTMS”). The term repetitive transcranial magnetic stimulation is often used interchangeably with the term transcranial magnetic stimulation in the clinical domain. Likewise, the abbreviation rTMS is often used interchangeably with TMS. Repetitive TMS has been tested as a treatment tool for various neurological and psychiatric disorders including migraines, strokes, Parkinson's disease, dystonia, tinnitus, depression, and auditory hallucinations.

TMS techniques typically act on a volume of brain tissue that is approximately two to three centimeters in diameter. The localized nature of the intervention avoids systemic side effects that commonly plague current pharmacologic treatments. This type of approach also avoids adverse medication interactions and the difficulty of ascertaining compliance with treatment as the patient must be physically present for treatment to occur.

As with most any medical treatments, currently known TMS techniques also entail potential side effects or risks, including headache or local scalp discomfort, hypomania in bipolar patients, and in rare cases seizure activity. A patient's hearing may also be adversely affected. During treatment, rapid deformation of the TMS coil produces a loud clicking sound that increases with the stimulator intensity. Such clicking can affect hearing with sufficient exposure. Consequently, hearing protection is typically used during TMS treatment.

C. Comparison of Techniques

Table 1 below compares some known electromagnetic neurostimulation techniques and illustrates certain characteristics of each. Note that the electromagnetic field emitted by these devices all have a non-zero electric field but may or may not have a non-zero magnetic field component.

TABLE 1
Comparison of Neurostimulation Techniques

| | | | | | Office | Brain |
|---|---|---|---|---|---|---|
| Stimulation | | Mag- | Non- | External | Proce- | Stimu- |
| Type | | netic | Invasive | Device | dure | lation |
| Vagus Nerve | VNS | NO | NO | NO | NO | NO |
| Stimulation | | | | | | |
| Peripheral | PNS | NO | NO | NO | NO | NO |
| Nerve | | | | | | |
| Stimulation | | | | | | |
| Deep Brain | DBS | NO | NO | NO | NO | YES |
| Stimulation | | | | | | |
| Cortical | CS | NO | NO | NO | NO | YES |
| Stimulation | | | | | | |
| Peripheral | PNFS | NO | NO | NO | YES | NO |
| Nerve Field | | | | | | |
| Stimulation | | | | | | |
| Transcutaneous | t- | NO | YES | YES | YES | NO |
| Vagal Nerve | VNS | | | | | |
| Stimulation | | | | | | |
| Transcutaneous | TENS | NO | YES | YES | YES | NO |
| Electrical Nerve | | | | | | |
| Stimulation | | | | | | |
| Electroconvulsive | ECT | NO | YES | YES | NO | YES |
| Therapy | | | | | | |
| Transcranial | tDCS | NO | YES | YES | YES | YES |
| Direct Current | | | | | | |
| Stimulation | | | | | | |
| Magnetic | MST | YES | YES | YES | NO | YES |
| Seizure | | | | | | |
| Therapy | | | | | | |
| Transcranial | TMS | YES | YES | YES | YES | YES |
| Magnetic | | | | | | |
| Stimulation | | | | | | |

In light of the above table and discussion, one of ordinary skill in the art understands that current neurologic and psychiatric treatments leave much to be desired. What are thus needed are novel systems and methods of treating neurologic or psychiatric disorders that are non-chemical, non-invasive, neuroplastic, and curative.

### SUMMARY

An exemplary embodiment of the disclosed subject matter is a therapeutic system comprising a brain stimulation device configured to stimulate a patient's brain by emitting an electromagnetic field based on certain stimulation parameters, a feedback device configured to measure data regarding brain activity, and a computer communicably connected to the feedback and stimulation devices. The brain stimulation device is preferably a non-invasive one. The computer is preferably configured to receive input from the feedback device and transmit an output to the brain stimulation device to adjust stimulation parameters dynamically. If there is input data indicating impending seizure activity, overheating, or pain, the output may comprise a signal that modifies the stimulation parameters to minimize side effects. The output may also comprise stimulation parameters designed to enhance or inhibit neuroplasticity in the patient's brain. The output may further comprise a signal designed to move or reorient the stimulation device to emit an electromagnetic field from a different location or to a different part of the patient's brain.

The brain stimulation device is preferably a transcranial magnetic stimulation device. The feedback device is preferably configured to perform quantitative electroencephalographic (“QEEG”) brain mapping, swLoreta brain imaging, or spectral analysis. The system preferably also includes a sensory stimulation device to provide sensory stimulation to the patient before, during, or after brain stimulation. The sensory stimulation device may be configured to deliver a variety of sensory stimulations depending on the disorder being treated. Sensory stimuli may include music, white noise, or sequenced tones individually notched for each ear for one or more of a patient's tinnitus frequencies; non-lyricized music or noise-cancellation headphones to treat auditory hallucinations; individually selected emotionally uplifting music to treat depression or for cognitive enhancement; or individually selected emotionally soothing music to treat generalized anxiety disorder (“GAD”) and post-traumatic stress disorder (“PTSD”).

Another exemplary embodiment of the disclosed subject matter is a method of therapeutic treatment comprising providing electromagnetic stimulation to a brain, and providing temporally associated sensory stimulation to treat a neurologic or psychiatric disorder or to enhance cognitive, motor, social, or psychological skills.

A further exemplary embodiment of the disclosed subject matter is a therapeutic method comprising providing electromagnetic stimulation to a brain using stimulation parameters, measuring effects of brain stimulation, and dynamically adjusting stimulation parameters to maximize treatment benefit.

Another exemplary embodiment of the disclosed subject matter is a method comprising determining a primary brain stimulation strategy(ies), optional secondary brain stimulation strategy(ies), and optional sensory stimulation strategy(ies); applying a primary brain stimulation strategy to effect brain stimulation; optionally applying a secondary brain stimulation strategy; optionally applying a sensory stimulation strategy before, during, or after primary or secondary brain stimulation; and optionally measuring effects of brain stimulation and adjusting stimulation parameters to maximize treatment.

Another exemplary embodiment of the disclosed subject matter is a method comprising determining a primary brain stimulation strategy using theta burst stimulation, optional secondary brain stimulation strategy using theta burst stimulation, and optional sensory stimulation strategy; applying the primary brain stimulation strategy to effect brain stimulation; optionally applying the secondary brain stimulation strategy; optionally applying the sensory stimulation strategy before, during, or after primary or secondary brain stimulation; and optionally measuring effects of brain stimulation and adjusting stimulation parameters to maximize treatment. Other alternatives to theta burst stimulation are alpha, beta, delta, or gamma burst stimulation depending on the frequency of the bursts within the pulse train.

One or more exemplary embodiments of the disclosed subject matter may also be understood to comprise dynamic, paired brain stimulation using individualized, maximally effective parameters based on specific regional brain activity to enhance neuroplastic changes in the human brain. The stimulation is of a specific brain region that is temporally paired with one or more sensory stimuli that may comprise visual, auditory, vestibular, haptic, gustatory, olfactory, pain, temperature, kinesthetic or other sensory stimulation patterns, secondary activities, thought processes, visual thinking, verbal thinking, emotions, medications, chemicals, physiological manipulations, neurofeedback, psychotherapy, videoconferencing, video recordings, social interaction, virtual reality (“VR”), guided imagery, electromagnetic brain stimulation directed at sensory cortical areas for the purposes of direct perceptual brain stimulation that bypasses peripheral sensory circuits, direct neural stimulation, brain-computer interface interaction, motor activities, sports activities, creative expression, art, musical expression, cognitive exercises, psychological exercises, meditation exercises, or other stimulation techniques designed to induce neuroplastic changes in targeted neuroanatomical substrates or circuits to modify neural wiring and thereby prophylax against or treat neurologic or psychiatric illnesses or enhance brain or body functioning.

One or more exemplary embodiments of the disclosed subject matter may further be understood to comprise direct or indirect TMS of the cingulate cortex (“CC”) or area of the brain with strong connections to the CC while paired with one or more specific sensory stimuli and in some cases preceded or followed by electromagnetic brain stimulation to the same brain area with different parameters or to another brain area. The paired sensory stimuli may include non-lyricized music or ambient noise cancellation for auditory hallucinations; emotionally uplifting music for depression or cognitive enhancement; notched music, notched white noise, or notched sequenced tones as stimulation for tinnitus; or individually selected emotionally soothing music to treat GAD and PTSD. Such sensory stimuli may be particularly paired with high frequency (e.g., 10, 20, or 30 Hz) or customized frequency of one hertz above the patient's individual alpha frequency (“IAF”), TMS of the left dorsolateral prefrontal cortex (“LDLPFC”) (approximate stimulation location electrode F3 position) or CC (midpoint of Cz, FC1, FC2), or RDLPFC (F4) and in some cases followed by an additional, diagnosis-specific TMS protocol. For example, to treat tinnitus the protocol may include low frequency stimulation of Brodmann Area 22 (midpoint between CP5 and T3 or CP6 and T4) contralateral to the side experienced with highest subjective volume or on the right side in cases of equal loudness. To treat depression, the protocol may include very high frequency stimulation (e.g., 20 Hz) of the LDLPFC at the junction of Brodmann Area 46 and 9 (F3). To treat GAD or PTSD the protocol may include inhibitory (e.g., low frequency 1 Hz) stimulation of the right dorsolateral prefrontal cortex (“RDLPFC”) at the junction of Brodmann Area 46 and 9 (F4). To treat auditory hallucinations, the protocol may include low frequency stimulation of Brodmann Area 39 in the left temporoparietal cortex (“TPC”) (CP5).

An additional exemplary embodiment of the disclosed subject matter is a method of neuroplastic augmentation using brain stimulation designed to augment, hasten, enhance, optimize, or improve a secondary neurologic or psychiatric treatment for a brain illness.

### BRIEF DESCRIPTION OF THE DRAWINGS

Some non-limiting exemplary embodiments of the disclosed subject matter are illustrated in the following drawings. Identical or duplicate or equivalent or similar structures, elements, or parts that appear in one or more drawings are generally labeled with the same reference numeral, optionally with an additional letter or letters to distinguish between similar objects or variants of objects, and may not be repeatedly labeled or described. Dimensions of components and features shown in the figures are chosen for convenience or clarity of presentation. For convenience or clarity, some elements or structures are not shown or shown only partially or with different perspective or from different point of views.

FIG. 1 is a perspective view of a patient being treated for a neurologic or psychiatric disorder, or treated to enhance cognitive, motor, social, or psychological skills, using a therapeutic system according to an exemplary embodiment of the disclosed subject matter;

FIG. 2A is a schematic overview of the exemplary system illustrated in FIG. 1;

FIG. 2B is a detailed schematic illustrating another exemplary system according to the disclosed subject matter;

FIG. 3 is a flow chart illustrating an overview of medical treatments according to exemplary embodiments of the disclosed subject matter;

FIG. 4A details exemplary aspects of the “patient's diagnosis, treatment needs” illustrated in FIG. 3 as certain psychiatric treatments;

FIG. 4B details exemplary aspects of the “patient's diagnosis, treatment needs” illustrated in FIG. 3 as certain neurologic treatments;

FIG. 4C details exemplary aspects of the “patient's diagnosis, treatment needs” illustrated in FIG. 3 as certain enhancement treatments;

FIG. 5 details exemplary aspects of the “primary brain stimulation strategy(ies), optional secondary brain stimulation strategy(ies), or optional sensory stimulation strategy(ies)” illustrated in FIG. 3;

FIG. 6A provides an overview in the context of psychiatric treatments and respective exemplary details applicable to “secondary brain stimulation strategy(ies)” and “temporally associated sensory stimulus(i)” illustrated in FIG. 3;

FIG. 6B provides an overview in the context of neurologic treatments and respective exemplary details applicable to “secondary brain stimulation strategy(ies)” and “temporally associated sensory stimulus(i)” illustrated in FIG. 3;

FIG. 6C provides an overview in the context of enhancement treatments and respective exemplary details applicable to “secondary brain stimulation strategy(ies)” and “temporally associated sensory stimulus(i)” illustrated in FIG. 3;

FIG. 7 is a flow chart illustrating certain dynamic treatment aspects according to exemplary embodiments of the disclosed subject matter;

FIGS. 8A, 8B, and 8C are brain images illustrating benefits of using exemplary embodiments of the disclosed subject matter; and

FIG. 9 is chart illustrating power versus frequency in the context of exemplary embodiments of the disclosed subject matter.

### DETAILED DESCRIPTION

A general problem in the field of brain disorders is treatments that are merely palliative, at best. A general solution is novel systems and methods of treating neurologic or psychiatric disorders that are curative.

A technical problem in the field of brain disorders is chemical-based or invasive treatments for psychiatric or neurologic disorders. A technical solution is novel systems and methods of treating neurologic or psychiatric disorders that are non-chemical and non-invasive.

Another technical solution implementing the spirit of the disclosed inventions is medical systems and methods for certain neurologic or psychiatric disorders using single-site and multi-site electromagnetic brain cell stimulation with temporally associated sensory stimulation.

Yet another technical solution implementing the spirit of the disclosed inventions is dynamic adjustment to brain stimulation parameters used during electromagnetic treatment.

Potential benefits of the general and technical solutions provided by the disclosed subject matter include not only curing psychiatric and neurologic disorders but also enhancing cognitive, psychological, social, or motor skills. The novel systems and methods herein achieve such benefits without the need of pharmacologic agents or invasive surgery. The disclosed systems and methods are also capable of real-time adjustments to stimulation parameters to maximize treatment benefit as well as preclude induced seizures.

A general nonlimiting overview of practicing the present disclosure is presented below. The overview outlines exemplary practice of embodiments of the present disclosure, providing a constructive basis for variant or alternative or divergent embodiments, some of which are subsequently described.

### I. Treatment Systems Overview

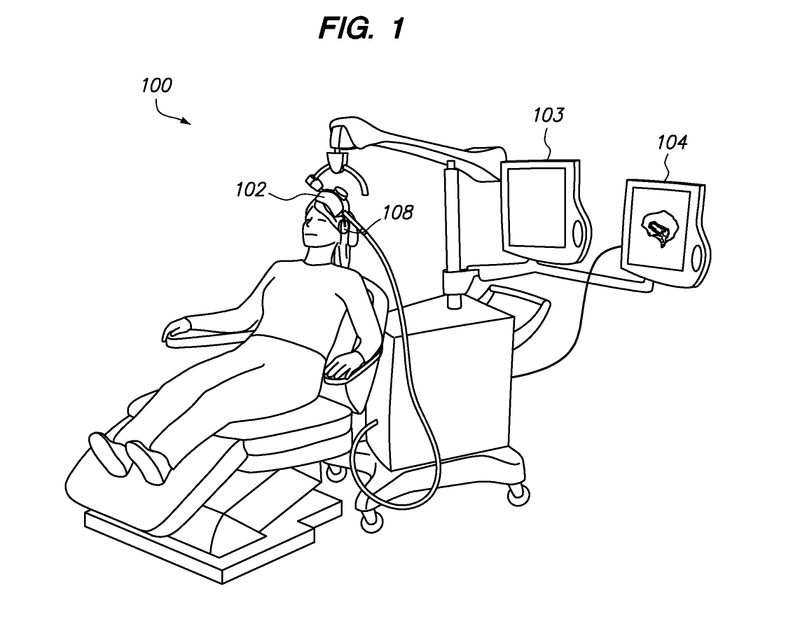

FIG. 1 illustrates an exemplary embodiment of a therapeutic system 100 comprising a brain stimulation device 102, a feedback device 104, a computer 106 communicably connected to the feedback and stimulation devices, and a sensory stimulation device 108 that may also be communicably connected to the computer 106.

The brain stimulation device 102 is configured to stimulate a patient's brain by emitting an electromagnetic field based on certain stimulation parameters. The stimulation device 102 is preferably a TMS device manufactured by Neuronetics, Inc. such as that of the NeuroStar® TMS Therapy System. The stimulation device 102 may also be a TMS device manufactured by The Magstim Company Ltd. such as the Magstim Rapid², Super Rapid², Super Rapid Plus¹, Magstim BiStim², and Magstim 200²; a TMS device manufactured by ANT B.V. such as the SmartMove; a TMS device manufactured by MagVenture A/S such as the MagPro®; a TMS device manufactured by Neotonus, Inc. such as the Neopulse Stimulator; a TMS device manufactured by Nexstim, Inc. such as the eXimia TMS Stimulator; or one or more similar such devices manufactured by Neuronix Ltd. (Israel), eNeuras Therapeutics (Sunnyvale, Calif.), or Neostim (San Mateo, Calif.). Likewise, the TMS device may be a patented device made by another manufacturer.

The brain stimulation device 102 may also be a transcranial direct current stimulation (“tDCS”) device such as the 1×1 tDCS or the 1×1 Limited Total Energy device or the 1×1 Clinical Trials stimulator. The tDCS device may be a product of Rogue Resolutions such as the neuroConn DC-Stimulator, the neuroConn DC-Stimulator Plus, the NeuroConn DC-Stimulator MR, or the neuroConn DC-Stimulator MC; or a product of Magstim such as the HDCkit, the HDCstim, or the HDCprog. The tDCS device may also be a high-definition tDCS device such as one manufactured by Soterix Medical, Inc. Likewise, the brain stimulation device may be a patented tDCS or HD-tDCS device made by another manufacturer.

Neuroplastic enhancement through direct or indirect brain stimulation of the CC is not limited to TMS, tDCS, or HD-tDCS. Other techniques include optical stimulation, ultrasound stimulation, and other stimulation techniques set forth above in Table 1. Any or all of these techniques may be used for direct or indirect stimulation of the CC while paired with ancillary stimuli to produce similar neuroplastic enhancement effected herein.

The feedback device 104 is configured to measure data regarding brain activity. The feedback device 104 is preferably configured to perform real-time QEEG brain mapping, cordance mapping as disclosed in U.S. Pat. No. 5,309,923, swLoreta brain imaging, or global frequency spectrum power. However, the feedback device 104 may also be configured for Loreta, sLoreta, magnetoencephalography (“MEG”), magnetic resonance imaging (“MRI”), near infrared spectroscopy (“NIRS”) diffusion tensor imaging (“DTI”), functional magnetic resonance imaging (“fMRI”), positron emission tomography (“PET”), single photon emission computer tomography (“SPECT”), nuclear magnetic spectroscopy (“NMS”), piezoelectric positional feedback, EMG, EKG, physiological parameters (HR, GSR, temperature, etc.), ultrasound, video camera, optical measurement device, or electrode potentials. Measurements obtained from the feedback device 104 are used to adjust stimulation parameters to maximize treatment benefit, including detailed mapping of the sensory cortex for phantom perceptual disorders. Known feedback devices 104 that may be used in one or more aspects of the exemplary embodiments include the neuronavigation devices manufactured by ANT B.V., such as the Visor or Visor-lite that includes brain computer interface (“BCI”) technology, the MagVenture neuronavigation system, or the Brainsight neuronavigation system.

The computer 106 is preferably configured to receive input from the feedback device 104 and transmit an output to the brain stimulation device 102. The computer 106 includes a central processing unit and at least one memory device that may coordinate the operation among the different parts of the system 100, as well as adjust stimulation parameters in real-time and deliver the output to the patient to enhance neuroplasticity in the patient's brain. For example, the computer may be configured to adjust TMS parameters such as intensity (expressed as percentage of motor threshold (“MT”)) until there is synchronous neural depolarization of the CC after the TMS pulse train. By doing so, custom parameter adjustments for each individual patient are obtained to realize a near 100% remission rate in response to TMS therapy in a variety of illness treatment contexts. As another example, the computer 106 may include one or more software algorithms that detect the active frequency for treating tinnitus disorders and modifies the TMS inhibitory stimulation so it is at a frequency that is not a harmonic of the hotspot.

The computer's output may also comprise a signal that modifies the stimulation due to input indicating coil overheating, significant scalp discomfort, or pre-seizure activity. For example, with real-time EEG monitoring, any potential seizure is going to be preceded by abnormal spike activity on EEG. Such activity is picked up during real-time monitoring. The computer 106 may be configured to include a software algorithm that continuously scans for seizure activity and applies seizure-specific inhibitory stimuli parameters or modifies the treatment parameters to low frequency (1 Hz) stimulation if pre-seizure waveforms begin to appear to suppress any seizure activity that may develop.

The output may further comprise a signal designed to move the brain stimulation device 102 to emit an electromagnetic field to a different part of the patient's brain or to emit the electromagnetic field from a different distance or orientation to the same part of the brain.

The sensory stimulation device 108 may provide sensory stimulation to the patient before, during, or after brain stimulation. The sensory stimulation device 108 may be configured to deliver a variety of sensory stimulations depending on the disorder being treated. Preferably, the patient should be paying attention to the sensory stimulation when applied.

Sensory stimuli may include music, white noise, or sequenced pure tones individually notched for each ear at a patient's tinnitus frequency; pure tone stimuli at the trauma frequency or in a notched pattern around the tinnitus frequency; the Dalton Stimulus for the suppression of tinnitus as discussed at http://www.wtamu.edu/news/joint-study-provides-advances-in-wtamu-tinnitus-research.aspx; silence or noise cancellation to treat auditory hallucinations; individually selected emotionally uplifting music to treat depression or enhance cognition; trauma-related virtual-reality stimulation to treat PTSD (with or without prior propranolol administration); haptic stimulation of specific dermatomes for treatment of chronic pain syndromes; low-intensity electrical stimulation of certain muscle groups; physical exercises; guided virtual-reality experiences or recorded video stimulation of athletic performances to enhance motor skills; guided mental exercise instructions to enhance cognitive skills; video-conferenced psychotherapeutic treatment (including cognitive behavioral therapy); guided imagery; guided meditation to enhance psychological skills; or guided simulations of social situations to enhance social skills or autism spectrum disorders.

Known sensory stimulation devices 108 that may be used in one or more aspects of the exemplary embodiments include headphones, monitors displaying video recordings, or virtual-reality devices or systems. The sensory stimulation device 108 may also include medications, chemicals, physiological manipulations, or other stimulation devices or techniques designed to induce neuroplastic changes in targeted neuroanatomical substrates or circuits.

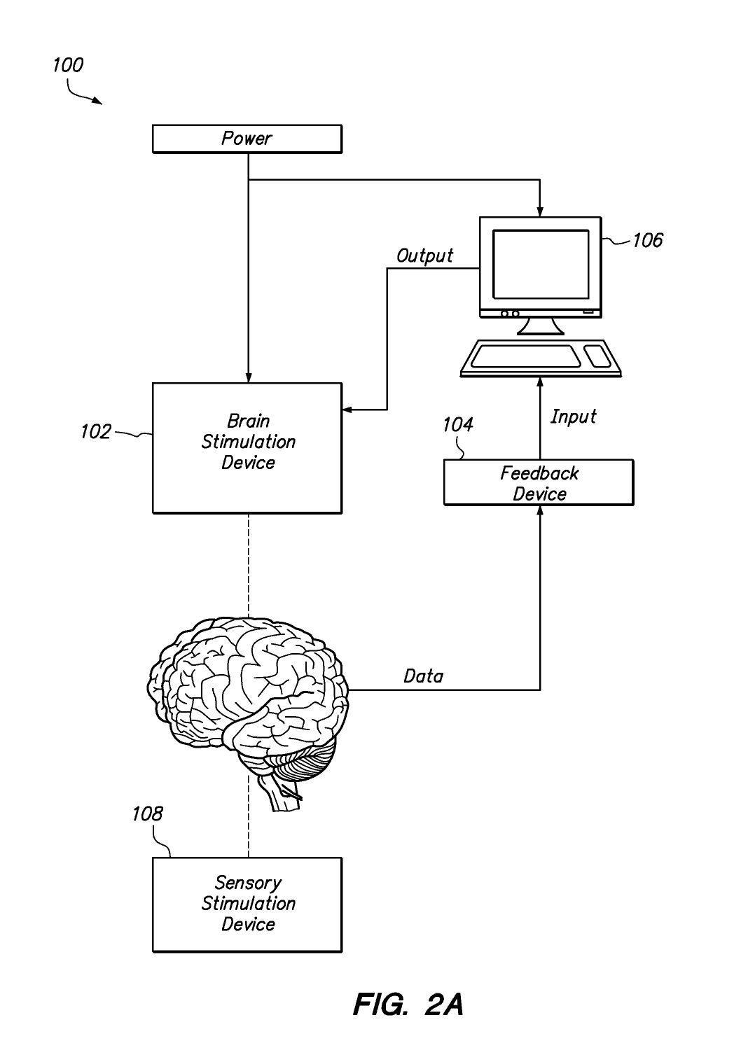

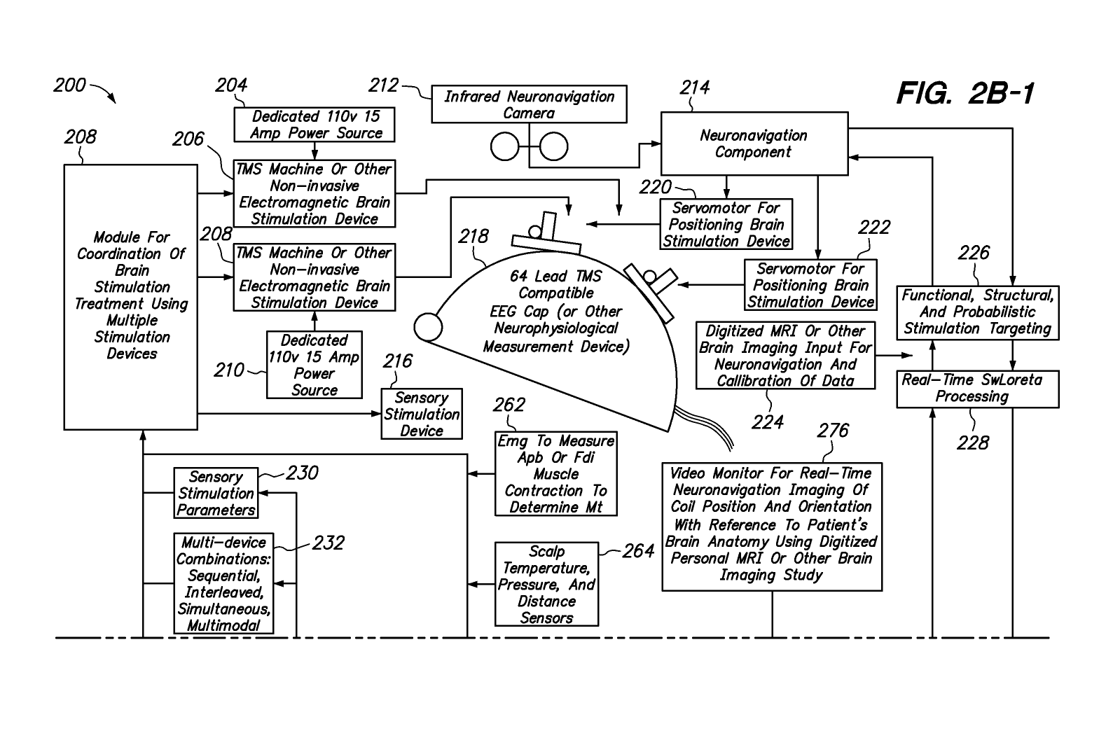

FIG. 2A is a schematic overview of the exemplary system 100 illustrated in FIG. 1. FIG. 2A particularly illustrates how measurement data is obtained from the brain by the feedback device 104. That data serves as input to the computer 106, which in turn generates an output to the brain stimulation device 102. The feedback device 104, computer 106, and brain stimulation device 102 may all be powered from the same power source. FIG. 2A also illustrates a sensory stimulation device 108 that may be configured to provide temporally associated sensory stimulation to treat a neurologic or psychiatric disorder or to enhance cognitive, motor, social, or psychological skills.

FIG. 2B is a detailed schematic illustrating another exemplary system according to the disclosed subject matter. As seen in FIG. 2B, treatment system 200 may include two or more brain stimulation devices 206, 208, which are preferably TMS devices or other non-invasive electromagnetic brain stimulation devices. Brain stimulation device 206 may be powered by its own dedicated 110 volt, 15 amp power source 204, whereas brain stimulation device 208 may be powered by another dedicated 110 volt, 15 amp power source 210.

A patient may receive electromagnetic stimulation from either or both brain stimulation devices 206, 208. Each brain stimulation device 206, 208 has its own respective servomotor 220, 222, for positioning each device 206, 208 about the patient's brain. Each servomotor 220, 222 may be positioned about a 64 lead TMS-compatible EEG cap or other neurophysiological measurement device 218.

Each servomotor 220, 222 may be communicably coupled to neuronavigation equipment such as an infrared neuronavigation camera 212 and neuronavigation component 214. The neuronavigation component 214 may receive functional, structural, and probabilistic stimulation targeting input 226 by way of real-time swLoreta processing 228; digitized MRI or other brain imaging input for neuronavigation and calibration of data, as illustrated by box 224 in FIG. 2B; and cordance analysis 290. The real-time swLoreta processing unit 228 may be in communication with a computer 282.

The computer 282 may be in communication with a wireless keyboard 284 and wireless computer printer 286. Computer 282 may also be in communication with a video monitor for a computer operating system 280; a video monitor for real-time cordance QEEG brain mapping, real-time swLoreta imaging, real-time power spectrum graphing, and ongoing raw EEG activity using preferred montage, as per box 278; and a video monitor for real-time neuronavigation imaging of coil position and orientation with reference to a patient's brain anatomy using digitized personal MRI or other brain imaging study, as per box 276.

Computer 282 may also be in communication with an amplifier 266, an analog to digital converter 268, a band pass filter 270, a notch filter 272, and an artifact removal component 274.

Computer 282 may be in further communication with a spectroscopic analysis component 288 and a module 208 for coordination of brain stimulation treatment using multiple stimulation devices, such as devices 206, 208. Module 208 may receive input including EMG to measure abducens pollicis brevis (“APB”) or first dorsal interosseus (“FDI”) muscle contraction to determine MT, as per box 262; and scalp, temperature, pressure, and distance sensors, as per box 264. Spectroscopic analysis component 282 may be in communication with cordance analysis 290, which in turn may involve MT percentage, as per box 250.

Module 208 may be in communication with sensory stimulation device 216 as well as MT percentage 250; frequency 248; stimulation timing 246; intertrain interval 244; stimulation interval 242; total number of pulses per session 240; if burst stimulation, then number of pulses per burst 238; pulse waveform shape 234; multi-device combinations including sequential, interleaved, simultaneous, or multimodal 232; and sensory stimulation parameters 230.

Spectroscopic analysis component 288 may also affect the delta power spectrum 258 including the delta band power spectrum and the alpha peak 260, which are involved with stimulation training 246 and frequency 248, respectively.

Computer 282 is also involved with an individualized diagnosis-specific treatment protocol plan and record, as per box 254, which may in turn be in communication with a printer for treatment records 252. Computer 282 may also be in communication with a pre-seizure detection component 256.

### II. Treatment Methods Overview

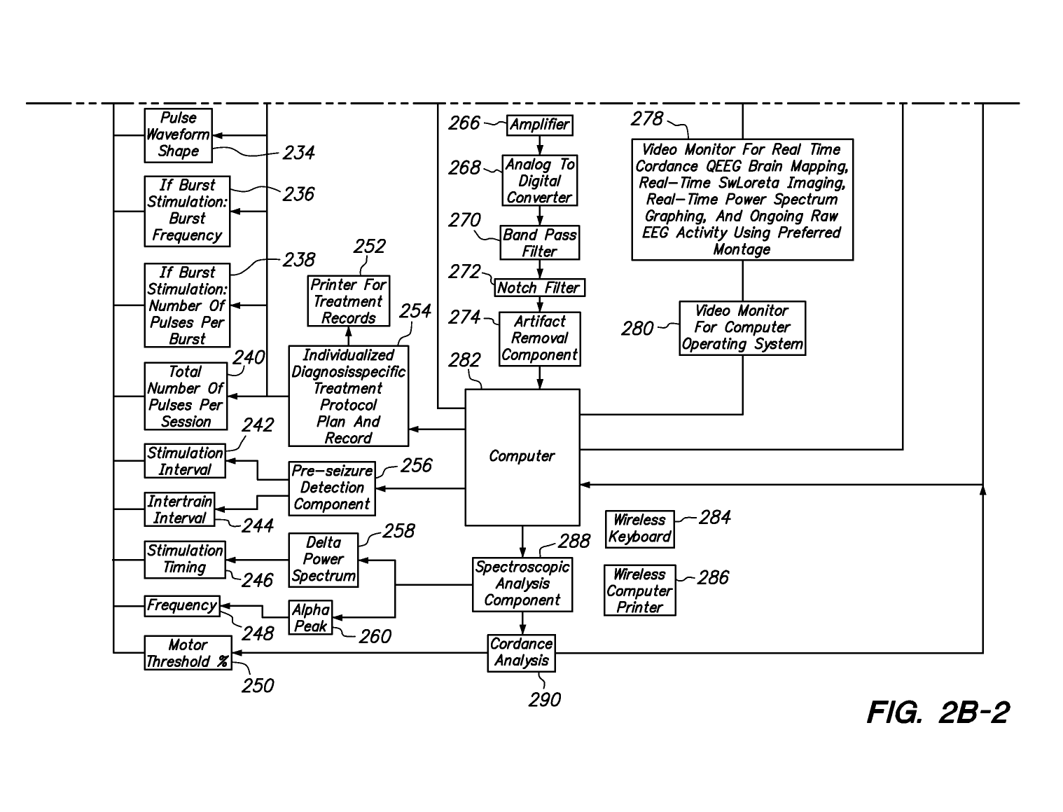

FIG. 3 is a flow chart illustrating an overview of medical treatments according to exemplary embodiments of the disclosed subject matter. In particular, FIG. 3 illustrates that the first step 110 may be directed to performing an initial diagnostic evaluation of the patient. This step 110 may include obtaining or reviewing an intake assessment, preliminary intake packet, Transcranial Magnetic Stimulation Adult Safety Screen Questionnaire (“TASS”), a copy of a chart from the referring doctor, past treatment records, digitized past imaging studies, copy of the last history and physical exam or specialist evaluation (e.g., ENT and audiology report for tinnitus), MRI, QEEG baseline, administered rating scales, contact notes with a referring doctor, private practice intake packet, consent form, structured clinical interview for DSM-IV form, or pre-treatment cordance values.

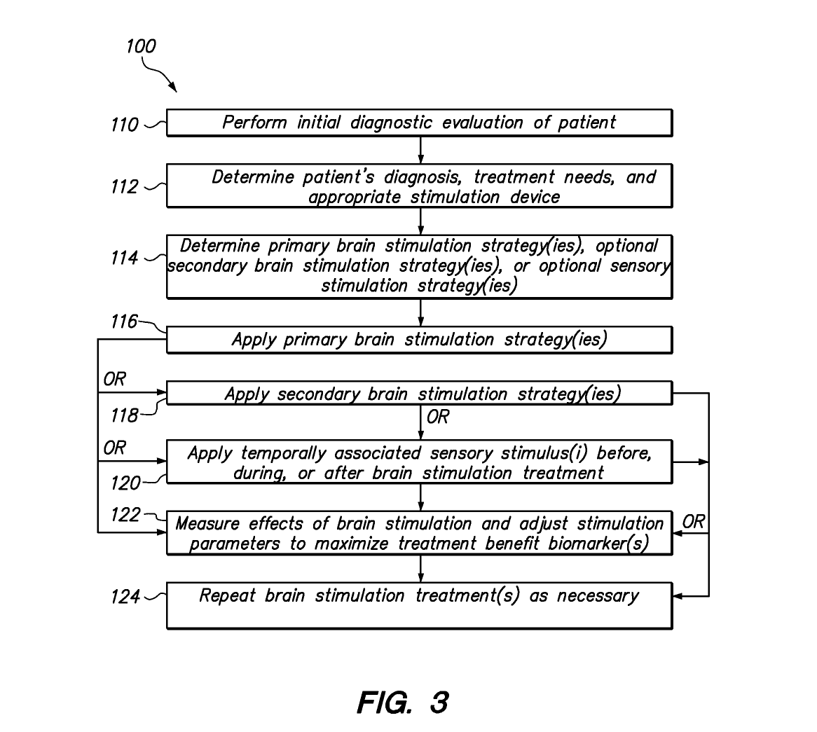

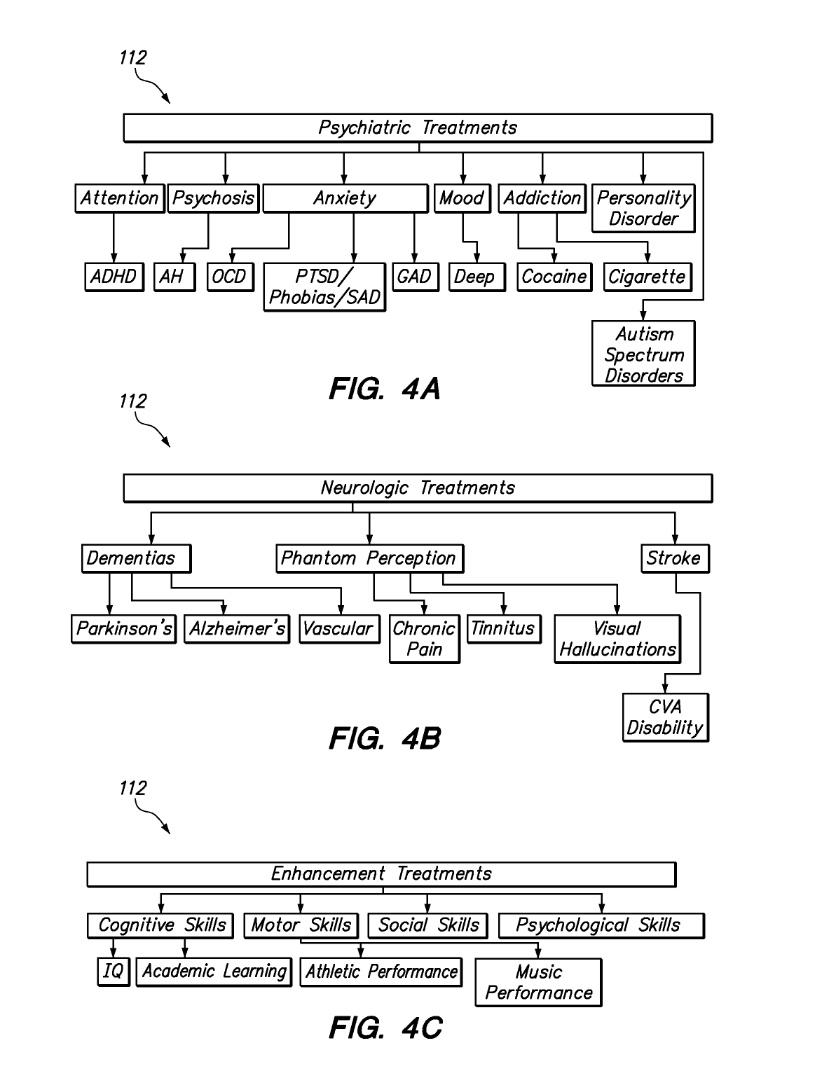

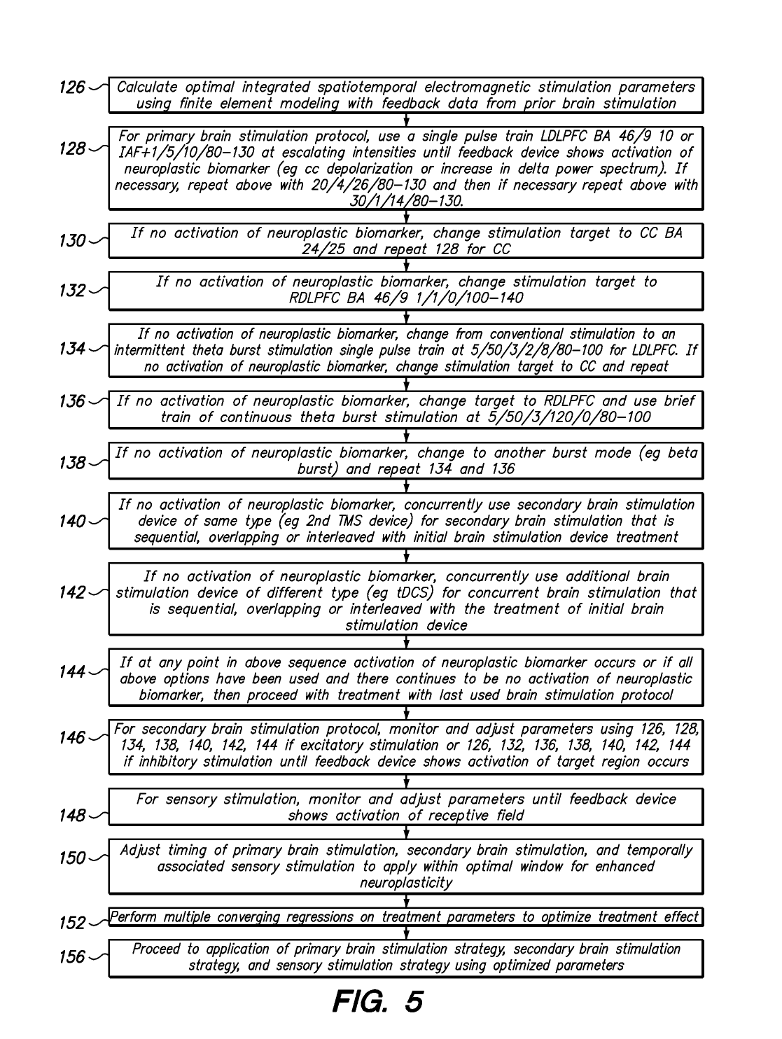

The next step 112 in an exemplary treatment method may include determining the patient's diagnosis, treatment needs, and appropriate brain stimulation device 102. Determining the patient's diagnosis and treatment needs may include determining whether the patient requires one or more psychiatric treatments, neurologic treatments, or both, depending on the diagnosed disorder(s), as well as determining whether the patient desires enhancement treatments. FIG. 4A illustrates certain psychiatric disorders treatable by the novel systems and methods disclosed herein, whereas FIG. 4B illustrates certain treatable neurologic disorders. FIG. 4C illustrates particular enhancement treatments that may be effected when using the disclosed systems and methods.

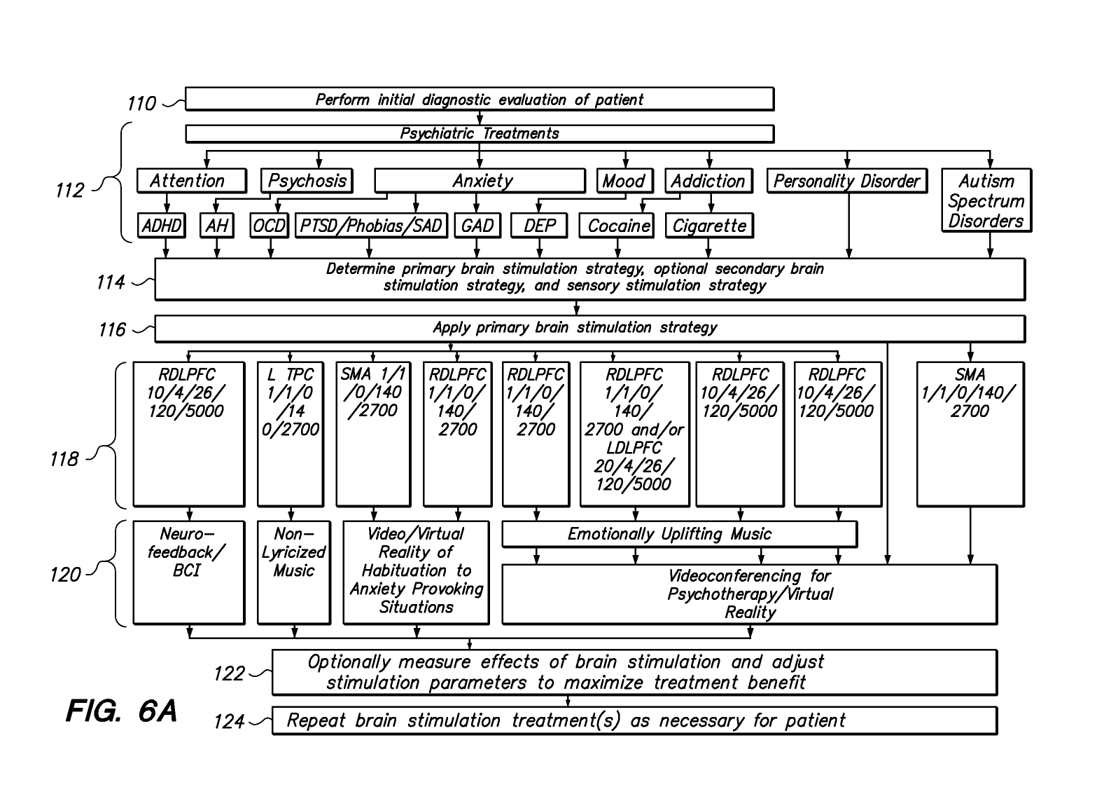

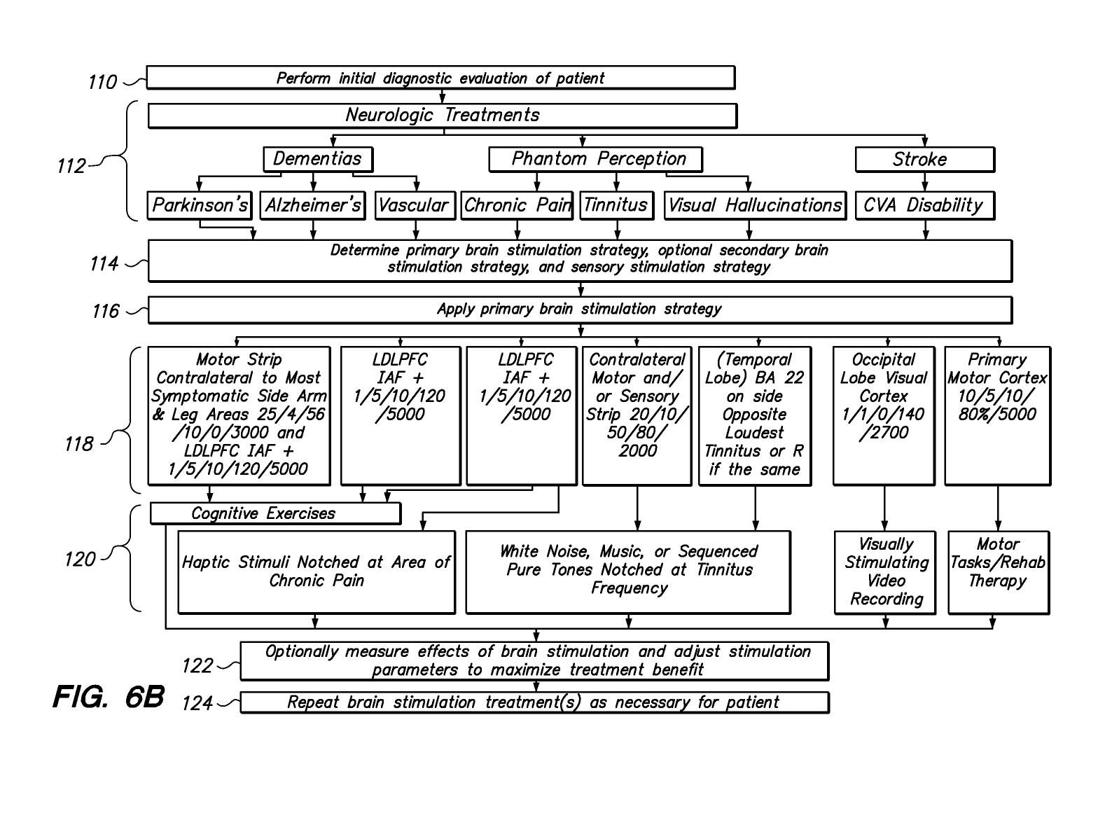

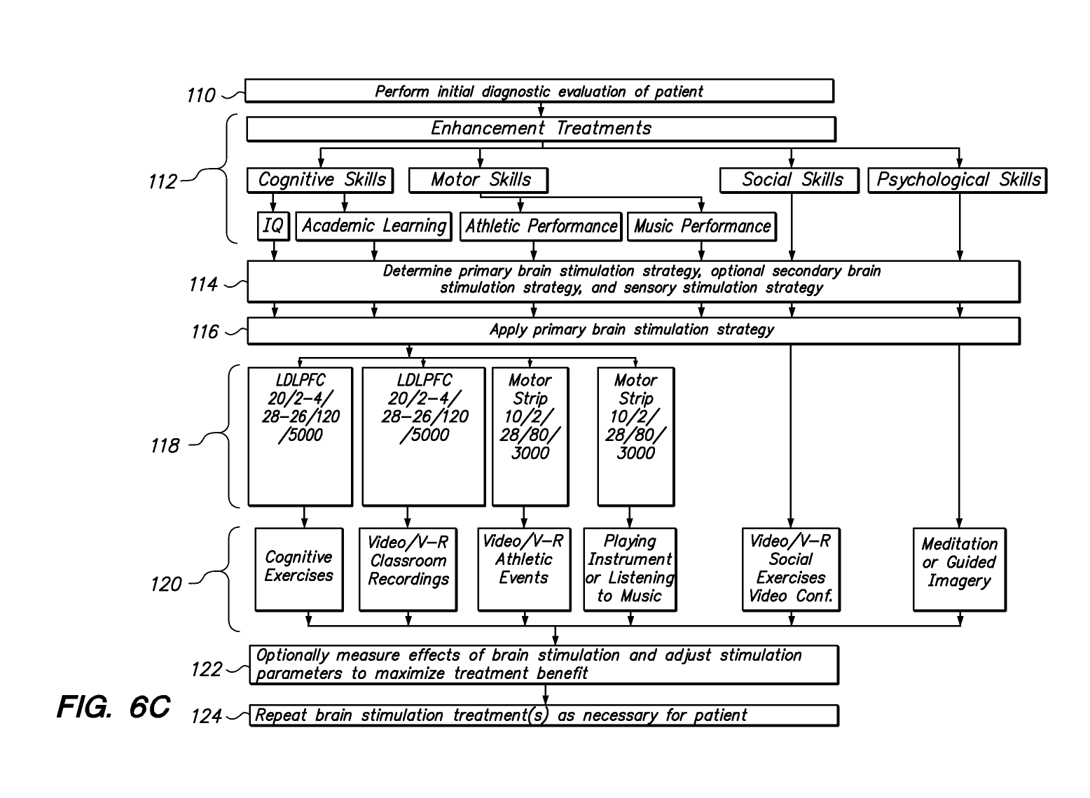

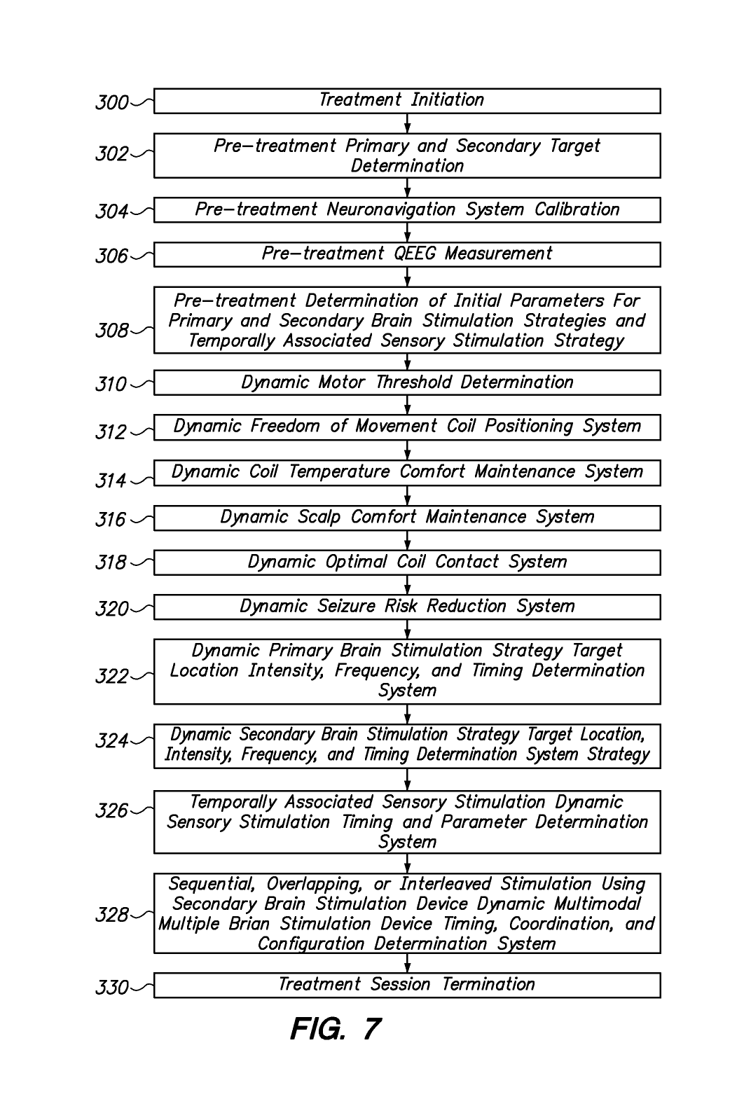

Turning back to FIG. 3, the next step 114 involves determining a primary brain stimulation strategy(ies), optional secondary brain stimulation strategy(ies), or optional sensory stimulation strategy(ies). Details of these strategies are discussed below in the context of FIGS. 5, 6A, 6B, and 6C. FIG. 3 also illustrates that there are optional steps thereafter, including the option of applying a secondary brain stimulation strategy(ies), per 118, which may optionally thereafter involve the steps of (1) applying a temporally associated sensory stimulus(i) before, during, or after brain stimulation treatment, per 120; or (2) measuring effects of brain stimulation and adjusting stimulation parameters to maximize treatment benefit biomarkers per step 122. As such steps are optional, the disclosed exemplary embodiments may include myriad different paths, as illustrated in FIG. 3, before arriving at the last step 124 involving repeating brain stimulation treatments as necessary. Preferably, in step 124 one should taper treatments by decreasing the periodicity of treatment until treatment ends or symptoms recur. If they recur, then one should begin maintenance treatment including periodic evaluation appointments, QEEG, and rating scales to monitor symptoms after ending treatment or during maintenance treatments.

### III. Primary Brain Stimulation Strategies

FIG. 5 details the “primary brain stimulation strategy(ies), optional secondary brain stimulation strategy(ies), or optional sensory stimulation strategy(ies)” illustrated in FIG. 3. For reference, the format of the parameter notation is x/y/z/a/b where x=stimulation frequency in Hertz, y=stimulation interval in seconds, z=intertrain interval in seconds, a=stimulation intensity as a percentage of MT, and b=total number of pulses for the treatment. If only three values are listed, they are x/y/z with the same definitions as above.

Step 126 may include calculating the optimal integrated spatiotemporal electromagnetic stimulation parameters using finite element modeling with feedback data from prior brain stimulation. This step 126 is followed by algorithmic steps 128 to 144 involved in determining the primary brain stimulation strategy. Before details regarding the steps 128 to 144 shown in FIG. 5 are discussed, some additional context is set forth here regarding brain disorders and stimulation techniques.

TMS triggers the release of dopamine (Keck et al. 2002; Pogarell 2006). Dopamine mediates human brain neuroplasticity (Thirugnanasambandam et al. 2010). Neuroplasticity is involved in the pathophysiology of depression (Castrén et al. 2009; Brunoni et al. 2008). Depression is linked to activation changes in the CC (Narushima et al. 2010; Pizzagalli et al. 2001; Stubbeman et al. 2004). CC is linked via frontocingulate circuits to the LDLPFC (Paus et al. 2001; Pizzagalli et al. 2011).

Stated differently, TMS releases dopamine, dopamine mediates neuroplasticity, neuroplasticity is critically involved in the pathology of depression, depression is connected to activation changes in CC, and the CC is linked via frontocingulate neural circuits to the LDLPFC. Combining these facts transitively, one aspect of the disclosed systems and methods herein is the recognition that TMS stimulation of the LDLPFC is a factor for enhancing neuroplasticity. Besides CC, other brain areas involved in neuroplasticity that may be measured to adjust stimulus parameters to maximize individual treatment efficacy include the frontal cortex, limbic system, amygdala, or hippocampus.

Returning to FIG. 5, primary brain stimulation step 128 may include using a single pulse train LDLPFC BA 46/9 10 or IAF+1/5/10/80-130 at escalating intensities until feedback device shows activation of neuroplastic biomarker (e.g., CC depolarization or increase in delta power spectrum). If necessary, repeat above with 20/4/26/80-130 and then if necessary repeat above with 30/1/14/80-130. In step 130, if there is no activation of neuroplastic biomarker, then change the stimulation target to CC BA 24/25 and repeat step 128 for CC. In step 132, if there is no activation of neuroplastic biomarker, then change the stimulation target to RDLPFC 1/1/0/100-140. In step 134, if there is no activation of neuroplastic biomarker, then change from conventional stimulation to an intermittent theta burst stimulation single pulse train at 5/50/3/2/8/80-100 for LDLPFC. If there is no activation of neuroplastic biomarker, then change the stimulation target to CC and repeat. In step 136, if there is no activation of neuroplastic biomarker, then change the target to RDLPFC and use brief train of continuous theta burst stimulation at 5/50/3/120/0/80-100. In step 138, if no activation of neuroplastic biomarker, then change to another burst mode, e.g., beta burst, and repeat steps 134 and 136. In step 140, if there is no activation of neuroplastic biomarker, then concurrently use a secondary brain stimulation device of the same type, e.g., a second TMS device, for secondary brain stimulation that is sequential, overlapping, or interleaved with initial brain stimulation device treatment. In step 142, if no activation of neuroplastic biomarker, then concurrently use an additional brain stimulation device of a different type, e.g., tDCS, for concurrent brain stimulation that is sequential, overlapping, or interleaved with the treatment of initial brain stimulation devices. In step 144, if at any point in the above sequence activation of neuroplastic biomarker occurs or if all above options have been used and there continues to be no activation of neuroplastic biomarker, then proceed with treatment with last used brain stimulation protocol.

After step 144, FIG. 5 illustrates that for secondary brain stimulation protocols, monitor and adjust parameters using steps 126, 128, 134, 138, 140, 142, 144 if excitatory stimulation or 126, 132, 136, 138, 140, 142, 144 if inhibitory stimulation until feedback device shows activation of target region. Next comes step 148 in the context of sensory stimulation protocols wherein one should monitor and adjust parameters until feedback device shows activation of receptive field. In step 150, adjust timing of primary brain stimulation, secondary brain stimulation, and temporally associated sensory stimulation to apply within optimal window for enhanced neuroplasticity. In step 152, perform multiple converging regressions on treatment parameters to optimize treatment effect. In the last step 154, proceed to application of primary brain stimulation strategy, secondary brain stimulation strategy, and sensory stimulation strategy using optimized parameters.

### IV. Optional Secondary Brain Stimulation and Paired Sensory Strategies

FIG. 6A provides an overview in the context of psychiatric treatments and details applicable “secondary brain stimulation strategy(ies)” 118 and “temporally associated sensory stimulus(i)” 120 illustrated in FIG. 3. FIG. 6B similarly provides an overview in the context of neurologic treatments and details applicable “secondary brain stimulation strategy(ies)” 118 and “temporally associated sensory stimulus(i)” 120 illustrated in FIG. 3. Finally, FIG. 6C provides an overview in the context of enhancement treatments and details applicable “secondary brain stimulation strategy(ies)” 118 and “temporally associated sensory stimulus(i)” 120 illustrated in FIG. 3.

Turning first to the psychiatric treatments in FIG. 6A, for attention disorders and particularly attention deficit disorders (“ADHD”), the preferred secondary brain stimulation strategy 118 is RDLPFC 10/4/26/120/5000; the preferred paired sensory stimulus 120 is neurofeedback or BCI. For the psychotic disorder of auditory hallucinations (“AH”), the preferred secondary brain stimulation strategy 118 is L TPC 1/1/0/14 0/2700; the preferred paired sensory stimulus 120 is non-lyricized music. For the anxiety disorder of obsessive compulsory disorder (“OCD”), the preferred secondary brain stimulation strategy 118 is SMA 1/1/0/140/2700; the preferred paired sensory stimulus 120 is video/virtual reality of habituation to anxiety provoking situations. For the anxiety disorder of PTSD/Phobias/SAD, the preferred secondary brain stimulation strategy 118 is RDLPFC 1/1/0/140/2700; the preferred paired sensory stimulus 120 is video/virtual reality of habituation to anxiety provoking situations. For the anxiety disorder of GAD, the preferred secondary brain stimulation strategy 118 is RDLPFC 1/10/140/2700; the preferred paired sensory stimulus 120 is emotionally uplifting music coupled with videoconferencing for psychotherapy.

For the mood disorder of depression, the preferred secondary brain stimulation strategy 118 is RDLPFC 1/10/140/2700 and/or LDLPFC 20/4/26/120/5000; the preferred paired sensory stimulus 120 is emotionally uplifting music coupled with videoconferencing for psychotherapy/virtual reality. For the addictive disorder of cocaine, the preferred secondary brain stimulation strategy 118 is RDLPFC 10/4/26/120/5000; the preferred paired sensory stimulus 120 is emotionally uplifting music coupled with videoconferencing for psychotherapy/virtual reality. For the addictive disorder of cigarettes, the preferred secondary brain stimulation strategy 118 is RDLPFC 10/4/26/120/5000; the preferred paired sensory stimulus 120 is emotionally uplifting music coupled with videoconferencing for psychotherapy/virtual reality. To treat a personality disorder, there is no preferred secondary brain stimulation strategy 118; however, the preferred paired sensory stimulus 120 is videoconferencing for psychotherapy/virtual reality. To treat autism spectrum disorders (“ASD”), the preferred secondary brain stimulation strategy 118 is SMA 1/1/0/140/2700; the preferred paired sensory stimulus 120 is videoconferencing for psychotherapy/virtual reality.

Turning now to the neurologic treatments in FIG. 6B, for the dementia disorder of Parkinson's disease, the preferred secondary brain stimulation strategy 118 is motor strip contralateral to the most symptomatic side arm and leg areas coupled with 25/4/56/100/3000 and LDLPFC IAF+1/5/10/120/5000; the preferred paired sensory stimulus 120 is cognitive exercises. For the dementia disorder of Alzheimer's disease, the preferred secondary brain stimulation strategy 118 is LDLPFC IAF+1/5/10/120/5000; the preferred paired sensory stimulus 120 is cognitive exercises. For the disorder of vascular dementia, the preferred secondary brain stimulation strategy 118 is LDLPFC IAF+1/5/10/120/5000; the preferred paired sensory stimulus 120 is cognitive exercises.

For the phantom perceptual disorder of chronic pain, the preferred secondary brain stimulation strategy 118 is contralateral motor and/or sensory strip 20/10/50/80/2000; the preferred paired sensory stimulus 120 is haptic stimuli notched at the area of chronic pain. For the phantom perceptual disorder of tinnitus, the preferred secondary brain stimulation strategy 118 may include (temporal lobe) BA 22 on side opposite the loudest tinnitus side or right side if the same; the preferred paired sensory stimulus 120 may include white noise, music, or sequenced pure tones notched at the tinnitus frequency(ies). Additional, complementary, and/or alternative exemplary embodiments pertinent to tinnitus are discussed in more detail below in Section V. For the phantom perceptual disorder of visual hallucinations, the preferred secondary brain stimulation strategy 118 is occipital lobe visual cortex 1/1/0/140/2700; the preferred paired sensory stimulus 120 is visually stimulating video recording.

To treat stroke and particularly CVA disability; the preferred secondary brain stimulation strategy 118 is primary motor cortex 10/5/10/80%/5000; the preferred paired sensory stimulus 120 is motor tasks/rehabilitation therapy.

Turning now to the enhancement treatments in FIG. 6C, to enhance cognitive skills and particularly a patient's intelligent quotient (“IQ”), the preferred secondary brain stimulation strategy 118 is LDLPFC 20/2-4/28-26/120/5000 and/or RDLPFC 1/1/0/120/2700; the preferred paired sensory stimulus 120 is EUM. To enhance cognitive skills and particularly a patient's academic learning ability, the preferred secondary brain stimulation strategy 118 is LDLPFC 20/2-4/28-26/120/5000 and/or RDLPFC 1/1/0/120/2700; the preferred paired sensory stimulus 120 is video or VR classroom recordings.

To enhance motor skills and particularly a patient's ability to increase athletic performance, the preferred secondary brain stimulation strategy 118 is motor strip 10/2/28/80/3000; the preferred paired sensory stimulus 120 is video or VR athletic events. To enhance motor skills and particularly a patient's ability to increase music performance, the preferred secondary brain stimulation strategy 118 is motor strip 10/2/28/80/3000; the preferred paired sensory stimulus 120 is playing an instrument(s) or listening to music.

To enhance social skills, there is no preferred secondary brain stimulation strategy 118, whereas the preferred paired sensory stimulus 120 is video or VR social exercises, or video conferences. To enhance psychological skills, there is no preferred secondary brain stimulation strategy 118; the preferred paired sensory stimulus 120 is meditation or guided imagery.

### V. Highlighted Treatment Methods

The following section highlights particular treatment methods according to exemplary embodiments in the context of certain disorders or in the context of neuroplastic augmentation of secondary brain treatments.

Phantom perceptual disorders such as tinnitus, chronic pain, phantom limb syndrome, as well as auditory hallucinations, are all thought to stem from the same underlying neurophysiology. The disorders are just manifested in different sensory cortical areas. To elaborate, in all of these disorders, there is decreased afferent input stimulation because of an initial trauma or insult.

The parts of our bodies that have the greatest density of touch receptors are on the sensory homunculus. If sensory input changes such that, for example, the thumb begins to be used more than it was before and the index finger begins to be used less, then the topologic sensory map for the thumb will grow larger. In contrast, the topologic sensory map for the index finger will contract in size, giving up some of its area to the thumb.

During normal plasticity changes due to increased or decreased sensory input on the sensory cortex, the sensitivity of the peripheral sensory receptor area increases or decreases in concert with the increase or decrease of the corresponding sensory field on the cortical topological map. Doing so will keep constant the ratio of peripheral neural input power to central cortical neurons corresponding to that particular sensory field. In contrast, in pathologic neuroplasticity that occurs in phantom perceptual disorders, the deafferented sensory field abruptly shrinks to keep constant the corresponding topologic cortical neurons and peripheral neural input power, the latter of which may be proportional to number of action potentials per unit area of cortex. However, because the surrounding sensory field expands to fill the void on the cortex of the brain, the ratio of input power to neurons in the surrounding area decreases, making these neurons compensate by decreasing their membrane potential that in turn increases their sensitivity. These hypersensitive neurons begin to fire spontaneously, giving rise to phantom perceptions in the absence of external stimuli. Even if the original deafferented area regains its afferentation, if neuroplasticity is impaired or if the traumatized area cannot increase its firing rate higher than the surrounding spontaneously hyperactive neurons, the phantom perceptions continue. The decreased area of sensory cortex corresponding to the traumatized sensory receptors becomes “trapped” by the surrounding hyperactive cortical area. Indeed, all phantom perceptual disorders such as phantom limb sensations, tinnitus, chronic pain, and auditory hallucinations appear to emerge from sudden deafferented sensory cortical nerves via similar pathological processes.

The applicable brain stimulation treatments disclosed herein involve selectively stimulating the traumatized area and other normally firing cortical regions, while leaving a “notch” in the pathologically hyperactive area. Thus, the surrounding sensory areas grow larger, their lateral inhibition returns, and the tonic hyperactivity abates. When this technique is temporally paired with neuroplastic enhancement, i.e., simultaneous LDLPFC stimulation, the malleability of the topology of the relevant sensory cortex increases. As a result, the treatment becomes accelerated and more effective.

For tinnitus, the disclosed treatment methods include temporally associated notched white noise to stimulate the whole tonotopic cortical map except for the tinnitus region, which has grown pathologically large. The pathologic area is “squeezed” back down to the original size, lateral inhibition increases, and the tonic hyperactivity in the frequency range of the tinnitus decreases. Other stimuli include “notched” pure tone stimulation of sensory areas outside the tinnitus area, including the original trauma frequency or music notched around the tinnitus frequency.

For some types of chronic pain, the disclosed treatment protocols stimulate motor cortex (counterintuitively, not sensory cortex) in the area of pain because afferent nerve tracts shrink in areas of chronic pain, whether through the initial trauma or lack of use. Thus, when one actively stimulates the area through motor neurons, the muscles surrounding the area of pain are activated. The subtle contractions are detected by sensory nerves that closely track muscle activity (essential feedback for learning motor skills), but there is a “notch” in the sensory stimulation in the area of pain because of the atrophied motor neuron input. As a result, the surrounding somatosensory map grows, the hyperactive region shrinks, the lateral inhibition from surrounding areas increases, and the tonic hyperactivity subsides.

For auditory hallucinations and schizophrenia, negative symptoms and neurocognitive deficits create a paucity of verbal thought. The brain areas responsible for verbal thinking (typically involving the left side of the brain for most people) are in a sense “deafferented.” These areas therefore contract and surrounding neurons involved in sensing language input become tonically hyperactive, leading the patient to hear voices. The disclosed treatment methods inhibit activity at this region, effectively “shrinking” the hyperactive area, allowing lateral inhibitory effects to take over, and diminishing and eventually stopping the auditory hallucinations.

For depression, studies have shown that there is often a generalized impaired neuroplastic capability of the brain (Normann et al. 2007). Furthermore, when a patient is treated with pharmacotherapy or ECT, the degree of improvement correlates with enhanced neuroplasticity (Chistyakov et al. 2005). For cognitive enhancement, there is also a generally enhanced neuroplastic capability of the brain, especially in the hippocampus where memories are formed via neuroplastic changes. Depression and cognitive ability may therefore be anatomically and symptomatically inversely correlated. As a result, when one uses the treatment systems and methods disclosed herein to treat depression, such use also measurably increases verbal fluency and memory capability, as well as measurably improves processing speed and visuospatial skills.

A. Tinnitus

Tinnitus affects about 10% of the global population, prevents 2% of the population from functioning either vocationally or socially, sometimes drives people to suicide, and is the leading cause of disability in soldiers coming back from recent wars. Tinnitus disability from veterans alone is costing the United States government billions of dollars a year.

Despite the severity of the problems associated with tinnitus, current treatments usually are considered a “success” if there is a 20% improvement in symptoms, and typically these treatments take six months to a year or involve brain surgery. In contrast, treating tinnitus according to exemplary embodiments of the disclosed subject matter yields dramatically improved results, including a 100% improvement in symptoms. Basically, patients are unexpectedly and miraculously cured when the disclosed novel systems and methods are employed.

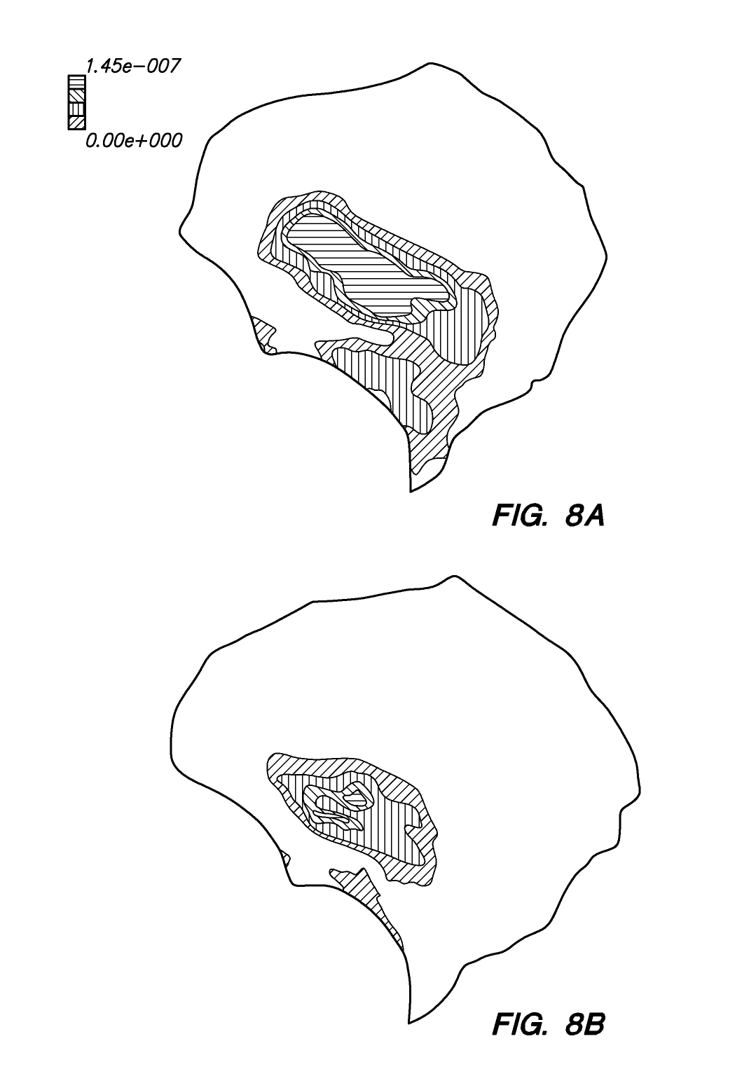

FIG. 7 illustrates an exemplary brain stimulation algorithm that may be performed in whole or in part by a software program, and one that may be particularly applicable for tinnitus. The initial step 300 involves treatment initiation, i.e., where one begins the TMS treatment session for tinnitus. Next comes pre-treatment target determination in step 302. In particular, step 302 involves marking the LDLPFC target location at the Brodmann Area 46/9 border at the middle third of the middle frontal gyri on the digitized MRI, and instructing the system to record Talairach coordinates of the LDLPFC target. Then mark the RDLPFC target location on the digitized MRI and instructs the system to record Talairach coordinates of the RDLPFC target. Next mark Brodmann Area 22 of the primary auditory cortex contralateral to the side of maximum perceived tinnitus volume and record Talairach coordinates. Then mark the Brodmann Area 22 location on the remaining side and record Talairach coordinates. Then mark Brodmann Area 25 of the CC bilaterally and record Talairach coordinates. Then mark the thumb knob of the motor strip bilaterally as a starting point for MT determinations. Next, activate the real-time video display of three-dimensional coil position referenced to brain anatomy by the neuronavigation system.

After pre-treatment target determination step 302 comes step 304 involving pre-treatment neuronavigation system calibration. In particular, in step 304, the patient is asked to sit in the chair and use any automatic controls to adjust the chair until the patient feels comfortable. Next, place the 64 lead EEG cap, such as cap 218 illustrated in FIG. 2B, on the patient's head and position the cap such that referenced distances from nasion, inion, and both pre-auricular spaces are in accord with the patient's individual reference values. Then calibrate the tracking system of the infrared neuronavigation camera, such as that illustrated by camera 212 in FIG. 2B, with the three-dimensional cluster of four infrared reflectors on the EEG cap, coil, reference pen, and coil calibration board.

After step 304 involving pre-treatment neuronavigation system calibration, the next step 306 is pre-treatment QEEG measurement. In particular, in step 306, perform a one-minute resting eyes-closed baseline QEEG and find the location of maximum weighted intensity of theta band activity in the region of interest. Then analyze the anterior, pregenual, and subgenual CC and mark the location on the digital brain image as the target. Next calculate the weighted cordance value in the region of interest and record target Talairach coordinates and weighted average cordance value in the patient's data file. Then display the patient's theta band swLoreta superimposed on the patient's digitized MRI on one side of a split-screen EEG video monitor; and display the patient's cordance brain maps on the other side of the split-screen EEG video monitor.

After step 306 involving pre-treatment QEEG measurement, the next step 308 is pre-treatment determination of initial parameters for primary and secondary brain stimulation strategies and temporally associated sensory stimulation strategies. The next step 310 is dynamic MT determination. In particular, in step 310, perform a new MT determination if (1) the MT was not obtained in the past week, (2) there is a new medication change, (3) the patient is sleep deprived, or (4) the patient has had caffeine before the procedure. A new MT is obtained using a dynamic electromyography system that (1) measures electrical activity in the patient's contralateral APB or FDI muscle after single pulse TMS treatment over the motor cortex and (2) graphs pulse location with muscular contraction intensity as measured by EMG on neuronavigation reconstruction of motor cortex surface anatomy. After the patient is comfortable and EMG electrodes are placed over the APB muscle of the contralateral hand, the system is instructed to perform a MT determination on one or both sides depending on laterality of treatment target locations. The system begins by placing a TMS coil over the projection of the hand knob of the motor cortex on the appropriate side for measurement. A test pulse of moderate intensity is triggered by the system after the coil is in place. The EMG value is recorded by the system. The coil is then moved 0.5 cm parallel to the axial plane and the procedure is repeated at a new location while the pulse intensity is held constant. The procedure is then repeated by the system by moving the coil position 0.5 cm parallel to the coronal plane. The procedure continues automatically in a grid pattern of stimulation points with a distance of 0.5 cm between points until a 3 cm by 3 cm grid search pattern has been performed on the cortical surface with the center over the anatomic landmark initially marked on the MRI. The Talairach coordinates of the cortical surface anatomy in three-dimensional space are superimposed on the APB contraction strength at each point represented by a scalar quantity in the region stimulated for the MT. The surface represented by a best-fit approximation created by a mesh is constructed and the local maxima and minima are calculated and marked digitally on the surface of the patient's brain. In other words, mark the point where the second derivative of the derived surface mesh is zero, and if there are multiple points fulfilling this requirement, the point is chosen that has the largest associated scalar quantity. This point is determined by the system to be the new MT location. The system then runs an algorithm of pulses at that location and measures the EMG response while coil stimulation intensity is now varied while the spatial location is held constant. After repeated measurements, the system determines the approximate coil intensity that triggers a thumb twitch of greater than 50 microvolts 50% of the time and a thumb twitch of less than 50 microvolts amplitude 50% of the time. That value is the newly derived MT intensity.

After step 310 involving dynamic MT determination, the next step 312 is dynamic freedom of movement regarding the coil positioning system. In particular, in step 312, activate the dynamic coil position to orient the servomotor feedback system so the calculated efield intensity is maintained at maximal value in the center of target volume Talairach coordinates, and the coil's position and orientation moves in real-time to maintain coil contact and orientation as the patient's head moves.

The next step 314 is dynamic coil temperature comfort maintenance system. Here, activate the dynamic coil temperature feedback system so a coil temperature reading is taken every 15 seconds and graphed continuously over time. A best-fit curve is fitted to the data points and extended until the projected end of the treatment session. If projected temperature versus time trajectory reaches a best-fit curve where the temperature is calculated to exceed 41 degrees Celsius, then the intertrain interval is automatically extended by intervals of one second until the projected best-fit curve does not exceed the temperature threshold. The intertrain interval value at that point is continued for subsequent pulse trains unless the projected temperature is again seen to rise above 41 degrees Celsius, at which point the procedure is repeated.

After step 314 comes step 316 involving dynamic scalp comfort maintenance system. In step 316, activate a dynamic scalp pressure feedback system so the patient's recorded preferred scalp contact pressure is maintained in real time without discomfort and measured in 100 millisecond intervals until 100 microseconds before a magnetic pulse is scheduled to fire. At that point, the measurement interval decreases to 1 microsecond intervals beginning 50 microseconds before pulse discharge, and the coil positioning system is switched to piezoelectric feedback system for 100 microseconds before pulse, 200 microseconds during pulse and 100 microseconds after pulse discharge, maintaining scalp pressure within desired target range. At 100 microseconds after magnetic pulse, the coil positioning system is taken over by servomotors until next magnetic pulse.

In step 318, activate the dynamic scalp distance feedback system so the coil face is never greater than a threshold distance, usually 1 mm, as measured by three micro-laser measuring devices embedded in the coil face. If the exceeds threshold distance from the scalp, either servomotor or piezoelectric positioning systems will be activated to close the distance. At the same time, a real-time feedback system is ongoing to keep an initial targeted ratio of three distance measurements constant so the coil is stable in all three rotational degrees of freedom if the infrared tracking system is unable to determine three-dimensional rotational position changes to the accuracy necessary to keep max efield continuously at the target location.

In step 320, activate a dynamic pre-seizure activity feedback system where real-time EEG is analyzed continuously and monitored for escalating clustered spike activity using a computerized seizure detection monitoring algorithm. If pre-seizure activity is detected, its principal focus is automatically calculated and the coil is moved immediately to that location. Pulse parameters then immediately change to continuous 1 Hz inhibitory treatment at 100% of the patient's MT as measured by monitored spike activity density. The treatment chair is automatically moved to a position nearest the floor to minimize possible trauma from a fall if a seizure does occur. Bilaterally, arm rests are raised to an elevated position to keep the patient in the chair if the patient becomes unconscious. Finally, recline the chair until the patient is in a supine position to protect the patient in case of a seizure. A warning bell is activated both locally in the treatment room and remotely at the front desk of a clinic to notify staff of possible impending seizure activity. If the spike density does not decrease after 5 seconds of inhibitory TMS treatment, MT % is increased at 5% intervals until spike activity begins to diminish. When preictal spike activity begins to diminish, one Hertz inhibitory stimulation is maintained at current MT intensity percentage until preictal spike activity vanishes. At that point, the coil arm is withdrawn, armrests are automatically lowered, the patient chair is elevated to a seated position so the patient may be evaluated by a treatment team, and the active treatment system is shut down but ongoing real-time EEG activity continues to be displayed on a screen to aid the treatment team in evaluating the patient's condition. If EEG spike activity moves from preictal to ictal and the beginning of a seizure is detected by the system, the coil arm is immediately withdrawn, and a more urgent auditory and visual alarm is triggered and emergency personnel are automatically called to the scene if the emergency procedure is not countermanded by staff. To prevent aspiration of mucus or vomit, the chair automatically tilts slightly by 15 degrees and the head rest rotates to turn the patient's head to the side in the direction the chair is tilted. The treatment chair arm on that side is further extended to give additional protection from falling in that direction. After the EEG cap is removed, the device automatically prints out a full report including EEG activity, pulse parameters, patient's treatment history, medication, etc. for reference for emergency personnel either on site or at the emergency room.

In step 322, activate a dynamic functional primary brain stimulation LDLPFC target location intensity, frequency, and timing determination system. The system uses the servomotor coil control system to move the coil so the maximum efield at the protocol-determined MT percentage intensity is centered on the LDLPFC target volume. A one-second duration at IAF+1 Hz (individual alpha frequency+1) train of pulses at 80% MT is administered and the weighted average theta-band (4-8 hz) cordance value in the region of interest defined to be anterior cingulate cortex including BA 25 is measured. The same search pattern as used before with MT is again applied, only instead of using the strength of EMG contraction, the CC cordance change from baseline is measured. Using an analogous procedure, the optimal target site on LDLPFC is determined by finding the site that maximally stimulates cingulate theta band cordance activity. Once the optimal target location of stimulation is found on LDLPFC, the intensity must be sufficient to trigger the neuroplastic changes necessary to restructure the pathologic patterns of brain illness. Brief one-second pulse trains are administered in 10% increments over 80% at IAF+1 looking for activation of CC area BA 25 current density followed by a significant decline or discharge of theta band energy. Once the intensity necessary for this activation is achieved, that intensity is used in the session for the LDLPFC treatment.

Next, the primary treatment coil is left in position over the LDLPFC, and the next step 324 is the dynamic targeting of the secondary treatment coil over the auditory cortex. The previously located target approximation on the right temporal cortex over BA 22 is where the system's servomotors move the secondary stimulation coil. The sensory stimulation system then emits a pure tone stimulus at the tinnitus frequency at a volume sufficient to overcome the tinnitus masking The swLoreta is then reconstructed looking for the area of maximum activation in the 30-50 Hz gamma frequency band. Once this location is determined, it becomes the dynamic target and the coil is automatically positioned by the system to focus the maximum efield on the area of the tonotopic map that is pathologically large and overactive. Next, continuous theta burst stimulation is administered to suppress the tinnitus frequency over the secondary auditory cortex. The intensity is elevated from 80-140% in 10% increments while observing the degree of decrease in activation intensity with different stimulation intensity. The intensity level where there is no further suppression of tinnitus activity is selected for this part of the treatment.

In step 326, one begins temporally associated randomized pure tone stimulation at the initial acoustic trauma frequency and at several discrete frequencies “notched” around the tinnitus frequency during the theta burst stimulus. The trauma frequency is distinct from the tinnitus frequency as the latter “crowds out” the trauma frequency on the tonotopic map. This technique causes high intensity activation of the auditory cortex in areas surrounding the tinnitus frequency while the inhibitory stimulus decreases the firing of the tinnitus during the peak plasticity window. Such actions have the effect of increasing the tonopic area of the trauma frequency that had been “squeezed” by the tinnitus frequency, and shrinking the tonotopic representation of the tinnitus frequency because it is especially vulnerable to the inhibitory treatment due to its high spontaneous firing rate.

Step 328 is directed to sequential, overlapping, or interleaved stimulation using a secondary brain stimulation device. In the context of treating tinnitus, in step 328, the treatment system is now poised for interleaved two-coil treatment of the tinnitus, where the LDLPFC coil stimulates for 5 seconds with an intertrain interval of 10 seconds, then the global frequency spectrum of the EEG is measured looking for a decrement and also a power peak higher than the usual alpha peak. When this configuration is achieved, the plasticity window is open and the secondary coil fires the continuous theta burst stimulus. Continuous theta burst stimulation is interleaved when the global spectral power has decreased below a certain threshold point, and if it rises above that point, the continuous theta burst pulses temporarily cease, preferably waiting until after the next LDLPFC stimulation at alpha+1 Hz. The system continuously monitors the individual alpha frequency and the LDLPFC frequency of stimulation is continuously updated so it is always one Hz higher than the individual alpha peak at that point in time. The interleaved stimuli are repeated until the number of pulses planned for the treatment session has been reached. These sessions are repeated daily on weekdays until the symptoms resolve.

In the final step 330 illustrated in FIG. 7, end the TMS treatment session for tinnitus.

Additional exemplary tinnitus treatment methods involve pairing notched music therapy with TMS stimulation of the LDLPFC. Immediately thereafter, inhibitory TMS stimulation may be employed over the auditory cortex contralateral to the side of the loudest tinnitus. If the loudness is equal bilaterally or it is too difficult for the patient to differentiate, the inhibitory stimulation may be applied over the right side. Because a patient may sometimes hear differing tinnitus frequencies on the right and the left, the music may be individually notched to the tinnitus frequency of each ear, meaning all sound frequencies within a narrow window frequency window called the “critical band” may be removed from the music using sound-mixing software (Adobe® Audition CS5.5). The music may be played using Bose® stereo headphones during the treatment with separate input channels for each earphone. The treatment location may be pin-pointed using commercially obtained neuronavigation equipment that coregistered each individual patient's MRI with infrared camera tracking technology to provide real-time visual imaging of TMS-induced electrical field intensities over individual neuroanatomical locations.

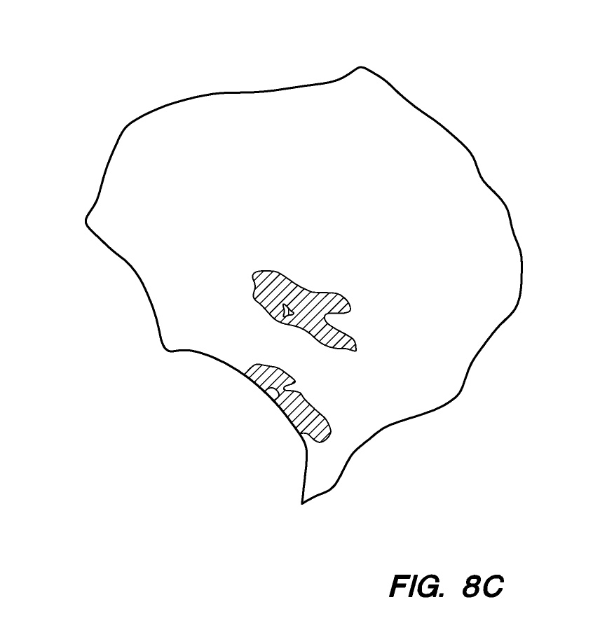

Brain activation over temporal cortex may be monitored weekly using swLoreta imaging of QEEG in the gamma frequency band (30-100 Hz) to track response. The notched music may be paired with LDLPFC stimulation resulting in concurrent improvement of left temporal cortex hyperactivity. In particular, due to implementation of the disclosed exemplary methods, the swLoreta brain images seen in FIGS. 8A, 8B, and 8C particularly reveal almost complete remission of tinnitus in over the course of several weeks of TMS treatment. The horizontal-cross-hatched areas seen in FIGS. 8A and 8B earlier in the treatment depict more intensive neural hyperactivity related to tinnitus compared to the other areas that show less activity (see the scale at far left of FIG. 8A) at points later in the treatment. Subjective reports of improvement paralleled the decreased activity on brain imaging and declining rating scale scores.

Further evidence of the efficacy of the disclosed embodiments was demonstrated when the notched music was stopped for two weeks while all other components of the treatment protocol remained the same (this occurred for a variety of clinical reasons, and was not intentionally done to verify treatment effectiveness). During this time the tinnitus worsened. Finally, when notched music therapy was again reinstituted, improvement rapidly resumed and was again reflected in tinnitus scales, brain-imaging, and subjective report.

Another exemplary tinnitus treatment method involves pairing notched sound therapy with TMS stimulation of either the LDLPFC, the CC, or RDLPFC followed by TMS stimulation to the auditory cortex the laterality of which had been determined before the treatment by a procedure called mismatch negativity (“MMN”) (Chung et al. 2012). During this procedure, a series of stimuli that change frequency very little if at all is unexpectedly followed by a sound frequency strikingly different from the initial monotonous tone sequence. By measuring the extent of brainwave alterations in both hemispheres in the auditory cortical areas, the side that demonstrates the greatest change is the dominant side for frequency processing; thus, the coil stimulus is applied to the corresponding side.

Another exemplary tinnitus treatment method involves using focused ultrasound stimulation of the LDLPFC followed by focused ultrasound stimulation of the auditory cortex opposite the loudest tinnitus side or right side if the same.

B. Auditory Hallucinations

Even in the most severe of cases and after all prior efforts fail, patients may be successfully treated for auditory hallucinations using the exemplary embodiments of the disclosed subject matter herein. The treatment protocol may include preliminary LDLPFC stimulation to facilitate neuroplastic changes. Specifically, the electrode location F3 may be used with the coil handle of a Neurostar® 2100 machine pointed posteriorly parallel to the transverse plane of the brain. Stimuli of 10 Hz may be applied for a 5-second stimulus interval alternating with a 10-second intertrain (pulse-free) interval at 95% of 0.4 Standard Motor Threshold (“SMT”) set artificially low due to limited tolerability for daily pulses.

This LDLPFC stimulation may be paired with non-lyricized music to target auditory neural circuits involved in sound perception including speech for enhanced neuroplastic malleability. This treatment may be followed by inhibitory treatment of the left SAC. The CP5 electrode location may be used with the coil handle angled inferiorly at a 45 degree angle from the sagittal plane. Continuous 1 Hz stimuli may be applied at 95% of 1.0 SMT for 1800 pulses. Using this approach, a patient's severe auditory hallucinations may be surprisingly eradicated completely.

By comparison, one of the largest studies of auditory hallucinations treated by TMS found 50% of patients showed an average of 50% improvement (Hoffman et al. 2005). A second large study found no statistically significant improvement in symptoms compared to sham (Slotema et al. 2010).

C. Bipolar I Depression, Bipolar II Depression, and Unipolar Depression

Patients with severe depression may also be successfully treated according to the disclosed exemplary embodiments. In particular, TMS was paired with LDLPFC stimulation with personally selected, emotionally uplifting music administered using headphones during TMS treatment of the LDLPFC. After obtaining informed consent, this uplifting music was added to the LDLPFC stimulation phase of TMS treatment. Patients were treated at the F3 location using one of three depression treatment protocols. Certain patients underwent the Hz stimulation protocol with a 5-second stimulation period, an intertrain interval at the minimum possible given the limits of the machine's capability but not less than ten seconds at customized percentages of MT within a range allowing for an antidepressant effect but not high enough to induce hypomanic symptoms for 5000-10000 pulses daily, 35000 pulses weekly during the acute phase of treatment. Other patients underwent the 20 Hz stimulation protocol with a 4-second stimulation period, 26-second intertrain interval, 120% MT for 5000 pulses daily. Certain other patients underwent the bilateral sequential protocol consisting of RDLPFC inhibitory TMS treatment combined with LDLPFC stimulatory treatment, the latter with the 10 Hz protocol. All patients had paired music stimulation with individually selected uplifting music. All patients dramatically improved and entered remission (full recovery) even though they had all lost hope of recovery until having treatment using the disclosed exemplary embodiments. Such a result is surprising considering that many of the treated patients had undergone multiple extensive medication trials for decades with no resolution of their symptoms, and some had also failed to improve with ECT.

By way of comparison, the two largest multicenter randomized controlled trials for TMS and depression treated over 500 patients. Both found that only 10-15% of patients remitted after TMS treatment, and these patients were much less ill than the patients discussed above (O'Reardon et al. 2007; George et al. 2010).

D. Generalized Anxiety Disorder

Patients with severe GAD may also be successfully treated using the disclosed embodiments. In particular, sequential RDLPFC inhibitory treatment may be followed by LDLPFC stimulatory treatment daily and paired with emotionally soothing classical music. Within three weeks, patients have been known to enter remission with dramatic improvements.

E. Post-Traumatic Stress Disorder

Even after years of unsuccessful multiple medication trials, patients with PTSD may also be successfully treated with the disclosed embodiments. In particular, sequential RDLPFC inhibitory treatment may be followed by LDLPFC stimulatory treatment administered in combination with psychotherapy to improve a patient's health dramatically.

F. Cognitive Enhancement

Patients who were treated for depression using the disclosed exemplary embodiments may also experience significantly enhanced cognitive skills. In particular, 10 Hz pulse trains for five seconds followed by at least fifteen-second intertrain intervals may be used to make dramatic improvements. After such treatment, patients may express how much easier it was to do crossword puzzles or the word jumble in the newspaper. Patients may report being able to read faster and absorb more. Patients may even use a noticeably larger vocabulary during the treatments.

G. Neuroplastic Augmentation of Secondary Brain Treatments

An additional exemplary embodiment of the disclosed subject matter is a method of neuroplastic augmentation using brain stimulation designed to augment, hasten, enhance, optimize, or improve a secondary neurologic or psychiatric treatment for a brain illness. Brain stimulation augments neuroplastic potential in one or more specific brain regions resulting in more timely or effective treatment of the brain disorder or fewer side effects than would otherwise have been the case for the neurologic or psychiatric treatment.

The brain stimulation treatment site may be determined by a feedback measurement device 104, such as QEEG cordance mapping. The feedback device 104 measures the area of interest that is preferably the region of peak electrical power output in midline CC theta band cortical activation (preferably in the electrode regions Cz, FC1, and FC2) or alternatively theta band swLoreta brain imaging of cingulate cortical activity (preferably in the region of Brodmann Areas 24 or 25).

Alternatively, the stimulation treatment site may be in a neuroanatomic region distinct from the CC that directly influences measured electrical power output in the CC due to connecting neural fiber tracts in the brain. The preferable remote stimulation site may be the LDLPFC at the junction of Brodmann Areas 46 and 9 near the midpoint of the left middle frontal gyms. In some instances, the brain stimulation frequency may direct the enhanced neuroplasticity to the specific brain region being treated by the secondary brain treatment modality. The brain stimulation frequency may be in the alpha band (most preferably at one Hertz above a patient's characteristic real-time alpha peak) when inducing plasticity changes in cognitive domains, the beta band (at 20 Hz) when desired plasticity changes occur in brain regions important in emotional expression, and gamma band when inducing plasticity changes in sensory cortices.

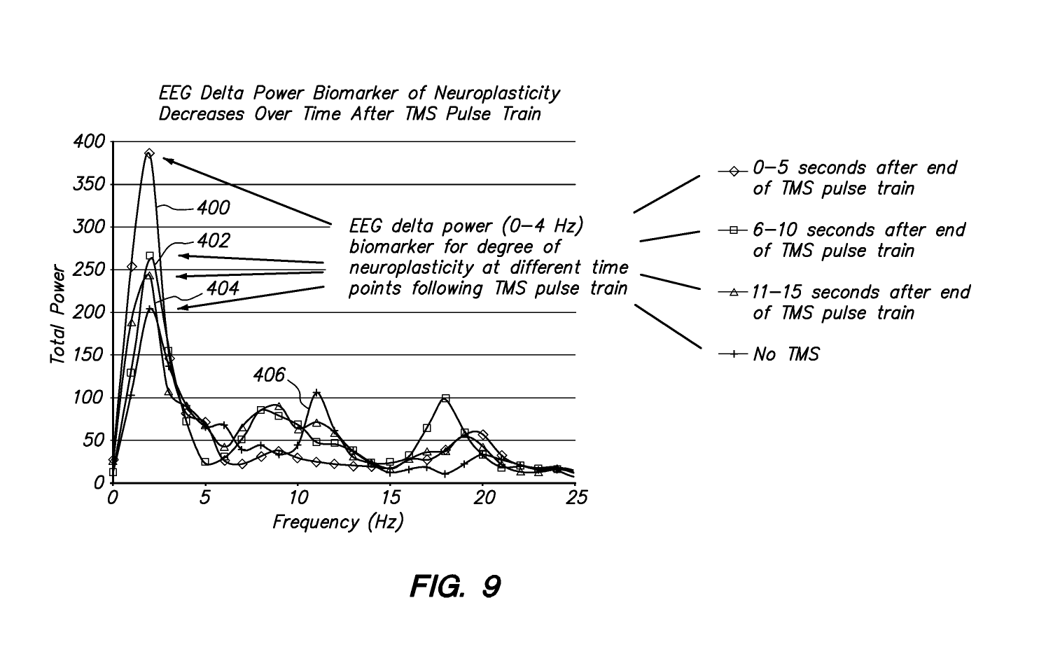

FIG. 9 nicely illustrates power versus frequency in the context of exemplary embodiments of the disclosed subject matter. Delta power has been shown to increase following administration of TMS (Gri\check{s}kova et al. 2007). EEG delta power has also been implicated as a biomarker for plasticity (De Gennaro et al. 2008).

FIG. 9 illustrates EEG delta power (0-4 Hz) and particularly shows differing amplitudes of delta wave power seen at different time points followed by a TMS pulse train. Each line corresponds to a latency period following a TMS pulse train. Line 400 illustrates 0-5 seconds after the end of the TMS pulse train. Line 402 illustrates 6-10 seconds after the end of the TMS pulse train. Line 404 illustrates 11-15 seconds after the end of the TMS pulse train. Line 406 illustrates no TMS. This figure reveals that the delta band power is greatest immediately after the neuroplastic activation of the pulse train and decreases over time as the window of plasticity closes.

The delta power plasticity biomarker in FIG. 9 indicates the import of precisely pairing the length and timing of one stimulus relative to another stimulus when administering brain stimulation. While it is difficult to measure the delta power spectrum during the pulse train itself because of the electromagnetic interference from the brain stimulator, the rapidly decreasing plasticity soon after the end of the pulse train reveals that the interval of plasticity began with or during the pulse train and ended soon after the pulse train ended. Thus, a paired sensory stimulus is preferably timed so as to be of approximately the same duration as the corresponding pulse train with the exception of a small phase delay. This phase delay maximizes the overlap of the sensory stimulus with the interval of greatest neuroplasticity activated by the brain stimulation pulse train. The precise degree of phase delay between the two stimuli is preferably constructed to benefit the treatment optimally.

After the initial electromagnetic brain stimulation, preferably the computer-linked feedback device 104 registers and quantifies the data using one of the QEEG measurement algorithms disclosed herein. If the brain electrical power density in the region of interest remains unchanged or increases, the computer may trigger another electromagnetic brain stimulation after a predetermined time interval. Most preferably, the process may be repeated until the electrical power activation level of the neurons in the region of interest decreases substantially, due in whole or in part to a coordinated release of neurotrophic factors that may act on the treatment region to facilitate and magnify the neural wiring changes occurring by the primary psychiatric or neurologic treatment. At that point, the brain stimulation augmentation portion of the treatment may cease and the secondary brain treatment may continue until completion.

The computer 106 may preferably contain an optimization algorithm wherein each of the treatment parameters is statistically correlated to the changes in activation level in the region of interest using an optimization protocol, preferably multivariate linear regression. With each repetition of the process, the treatment parameters may be systematically varied to determine the coefficients of multiple determination for each of the principle treatment parameters of the brain stimulation device 102. Each iteration of electromagnetic stimulation of the target area may have modified parameters in accordance with the results of the statistical regression analysis so the electromagnetic stimulation of the target area may become more and more effective over the duration of the brain stimulation neuroplasticity modulation element of the psychiatric or neurologic treatment.

The brain stimulation neuroplasticity modulation technique may occur before, during, or after the neurologic or psychiatric treatment depending on the specific details of the treatment protocol and the output measurements of the feedback device 104. For example, when the secondary psychiatric or neurologic treatment is also a brain stimulation treatment such as ECT, the neuroplastic modulation may occur just before the emission of the current charge. When the augmented psychiatric or neurologic treatment is a medication treatment, the neuroplastic modulation may occur subsequent to ingestion of the medication. When the augmented psychiatric or neurologic treatment is a form of speech therapy due to a neurologic insult, the neuroplastic modulation may temporally coincide with the administration of the speech therapy training

H. Burst Stimulation Treatment

Patterned or burst stimulation protocols in brain stimulation treatment were introduced into the literature in 2005. They were initially used for research into cortical plasticity because they induced long-term neuroplastic changes in cortical neurons that greatly outlasted the stimulation duration. Burst stimulation simply substitutes a cluster of two or more high-frequency, rapid-fire pulses in the place of a single pulse of conventional TMS. The most common type of burst protocol is called theta burst stimulation (“TBS”). It consists of 5 Hz (theta band is 4-8 Hz) TMS stimulation where in place of every single pulse of 5 Hz conventional stimulation, three pulses are substituted that have a frequency of 50 Hz, i.e., three pulses separated by 20 milliseconds and each cluster is separated from the other clusters by 200 milliseconds.

Perhaps because this type of stimulation pattern was modeled on neuroplastic firing patterns that occur naturally in the brain, theta burst stimulation has been found to cause neural changes that last much longer than conventional brain stimulation protocols. When theta burst protocols are substituted for conventional TMS protocols, there is often a greater degree of response in a shorter amount of time, and the response is more sustainable.

The exemplary embodiments disclosed herein may use theta burst stimulation as the secondary brain stimulation strategy, preceded by notched auditory stimuli (such as white noise or scattered pure tones) temporally associated with conventional 10 Hz LDLPFC TMS stimulation to cure tinnitus. Indeed, as a result of a single neuronavigated theta burst treatment lasting less than two minutes, patients found their completely unacceptable tinnitus frequencies to be inaudible.

Quadripulse burst protocols have been successfully tested that contain four high frequency pulses in a cluster rather than three. Also other types of burst protocols have been given that involve a burst frequency of delta (0-4 Hz), alpha (8-12 Hz), beta (12-30 Hz), and gamma (30-100 Hz) frequencies. Burst protocols may have an arbitrary number of high-frequency pulses in each cluster ranging from 2-100 pulses per cluster or more, limited only by physics and the output capabilities of the stimulation device.

Generally burst protocols fall into stimulatory or inhibitory categories. An example of an inhibitory type of stimulation is continuous theta burst stimulation (“cTBS”) where bursts of three pulses at 50 Hz are applied at a frequency of 5 Hz for a total of 100-400 bursts or more in a treatment session. An example of an excitatory type of burst stimulation is intermittent theta burst (“iTBS”), which consists of two-second periods (10 bursts with a total of 30 pulses) that are applied at a rate of 0.1 Hz for a total of for a total of 20, 30, or more two-second periods.

Because of theoretical and practical success with burst protocols, brain stimulation methods described in these treatment methods include both conventional and burst protocols as well as other forms of patterned pulse stimulation. Anywhere in the disclosed treatment methods where excitatory conventional brain stimulation in the 5-30 Hz or higher range is given as a possible treatment protocol for neurological, psychiatric, or enhancement therapy, it is understood that a burst protocol may just as easily be substituted with intermittent theta burst stimulation, for example. Likewise, anywhere in these treatment methods where an inhibitory stimulation in the 0-5 Hz range is described, it is understood that a burst protocol may just as easily replace it with continuous theta burst, for example. A cluster of pulses may be substituted for a single pulse in a conventional treatment strategy, or a burst protocol consisting of totally unrelated parameters may be substituted for conventional brain stimulation as long as it may give a positive treatment outcome. These burst patterns, including but not limited to theta burst stimulation, have a profound effect on the ability of brain stimulation to treat a variety of neurological and psychiatric illnesses successfully and further enhance cognitive, motor, social, or psychological skills to allow an individual to reach their maximum potential in one or more domains of functioning.

An exemplary embodiment of the treatment method using burst protocols for the relief of tinnitus is as follows. A primary brain stimulation strategy for tinnitus may consist of transcranial magnetic stimulation to the LDPFC with high frequency conventional stimulation (5-30 Hz or higher) or alternatively with an iTBS protocol consisting of a total of 20 two-second periods that is temporally associated with auditory sensory stimulation consisting of music, white noise, or pure tone sequences notched around the tinnitus frequency. This stimulation may be followed by a secondary brain stimulation strategy of inhibitory brain stimulation to Brodmann Area 22 that overlies the primary auditory cortices (BA 41 and 42). The inhibitory stimulus may also be applied in secondary or tertiary auditory cortical processing regions. The inhibitory stimulus may be applied on the side opposite that of the loudest perception of tinnitus volume, or on the right side if the two sides are equally loud, or in some cases on both sides, or in other cases using a mismatch negativity measurement that has been determined beforehand by EEG (or MMNm in the case of an MEG) measurement after a novel acoustic frequency stimulus is placed in a long sequence of very similar frequency stimuli that triggers a large(r) alteration of brain wave activity in the particular hemisphere in which sound frequency processing is greatest. The inhibitory stimulation may consist of cTBS for 400 bursts (1200 total pulses). This type of stimulation may be repeated five days a week for 4-8 weeks or however long it takes for the tinnitus to resolve. This stimulation protocol may also be used in conjunction with the dynamic feedback system described herein so a feedback device adjusts the stimulation parameters in real time based on the output of a measurement device contained in the system (preferably a measurement device assessing CC activity and modifying stimulus parameters to depolarize the brain region after a treatment session to induce neuroplastic release of neurotrophic factors or otherwise facilitate a neuroplastic response). Another possible additional component of this protocol may include the concomitant use of transcranial direct current stimulation to facilitate or inhibit as appropriate the TMS of the primary or secondary treatment strategies. This combination may be especially synergistic because transcranial direct current stimulation does not normally trigger action potentials in the target neurons. It simply increases or decreases their tendency to fire presumably by altering the neural transmembrane potential. On the other hand, TMS generally directly triggers at least some amount of neural firing. By combining the two modalities, neural pathways are “primed” by transcranial direct current stimulation to be more sensitive to TMS. One of many possible alternatives to the above exemplary embodiment may be the use of (preferably stimulatory) CC brain stimulation applied at or near the midline or (preferably inhibitory) RDLPFC stimulation that may have cortico-cortical connections to the CC via a more circuitous pathway than the cortico-cortical connections between LDLPFC and CC.

While certain embodiments have been described, the embodiments have been presented by way of example only and are not intended to limit the scope of the inventions. Indeed, the novel devices and methods described herein may be embodied in a variety of other forms; furthermore, various omissions, substitutions, and changes in the form of the devices and methods described herein may be made without departing from the spirit of the inventions. For example, techniques, systems, subsystems, and methods described and illustrated in the various embodiments as discrete or separate may be combined or integrated with other systems, modules, techniques, or methods without departing from the scope and spirit of the present disclosure. Other items shown or discussed as coupled or directly coupled or communicating with each other may be indirectly coupled or communicating through some interface, device, or intermediate component whether electrically, mechanically, or otherwise. The accompanying claims and their equivalents are intended to cover such forms or modifications as would fall within the scope and spirit of the inventions.

Indeed, none of the description in the present application should be read as implying that any particular element, step, or function is an essential element that must be included in the claim scope. In contrast, the scope of the patented subject matter is defined only by the allowed claims. Moreover, none of the claims is intended to invoke paragraph six of 35 U.S.C. section 112 unless the exact words “means for” are followed by a participle. The claims as filed as intended to be as comprehensive as possible, and no subject matter is intentionally relinquished, dedicated, or abandoned.

Moreover, the texts and drawings of the Appendix are incorporated into the application by reference as an integral part of the application. Additionally, the documents listed in the Appendix are incorporated into the application by reference.

### APPENDIX

### List of Citations

1. Brunoni et al., “A Systematic Review and Meta-Analysis of Clinical Studies on Major Depression and BDNF levels: Implications for the Role of Neuroplasticity in Depression,” The International Journal of Neuropsychopharmacology 11:1169-1118 (2008).
2. Castrén et al., “The Role of BDNF and its Receptors in Depression and Antidepressant Drug Action: Reactivation of Developmental Plasticity,” Developmental Neurobiology 70: 289-297 (2010).
3. Chistyakov et al., “Antidepressant Effects of Different Schedules of Repetitive Transcranial Magnetic Stimulation vs. Clomipramine in Patients with Major Depression: Relationship to Changes in Cortical Excitability,” International Journal of Psychopharmacology 8:223-233 (2005).
4. Chung et al., “Effectiveness of Theta-Burst Repetitive Transcranial Magnetic Stimulation for Treating Chronic Tinnitus,” Audiology and Neurotology 17:112-120 (2012).
5. De Gennaro et al., “Cortical Plasticity Induced by Transcranial Magnetic Stimulation During Wakefulness Affects Electroencephalogram Activity During Sleep,” PLoS ONE 3(6): e2483 (2008).
6. Doidge, The Brain That Changes: Itself Stories of Personal Triumph From the Frontiers of Brain Science (2007).
7. Engineer et al., “Reversing Pathological Neural Activity Using Targeted Plasticity,” Nature 470:101-104 (2011).
8. George et al., “Daily Left Prefrontal Transcranial Magnetic Stimulation Therapy for Major Depressive Disorder: A Sham-Controlled Randomized Trial,” Archives of General Psychiatry 67(5):507-516 (2010).
9. Gri\check{s}kova et al., “The Effects of 10 Hz Repetitive Transcranial Magnetic Stimulation on Resting EEG Power Spectrum in Healthy Subjects,” ScienceDirect 162-167 (2007).
10. Hatton et al., “Vagal Nerve Stimulation: Overview and Implications for Anesthesiologists,” Anesthesia & Analgesia 103:1241-1249 (2006).
11. Hoffman et al., “Temporoparietal Transcranial Magnetic Stimulation for Auditory Hallucinations: Safety, Efficacy and Moderators in a Fifty Patient Sample,” Journal of Biological Psychiatry 58:97-104 (2005).
12. Hsieh et al., “Sleep-Related Breathing Disorder in Children with Vagal Nerve Stimulators,” Pediatric Neurology 38:99-103 (2008).
13. Keck et al., “Repetitive Transcranial Magnetic Stimulation Increases the Release of Dopamine in the Mesolimbic and Mesostriatal System,” Neuropharmacology 43:101-109 (2002).
14. Kessler et al., “Prevalence, Severity, and Comorbidity of 12-Month DSM-IV Disorders in the National Comorbidity Survey Replication,” Archives of General Psychiatry 62:617-627 (2005).
15. Kim et al., “Prevalence of Autism Spectrum Disorders in a Total Population Sample,” American Journal of Psychiatry 168:904-912 (2011).
16. MacDonald et al., “The Incidence and Lifetime Prevalence of Neurological Disorders in a Prospective Community-Based Study in the UK,” Brain 123:665-676 (2000).
17. Mantyselka et al., “Chronic Pain and Poor Self-Rated Health,” Journal of the American Medical Association 290(18):2435-2442 (2003).
18. Narushima et al., “Subgenual Cingulate Theta Activity Predicts Treatment Response of Repetitive Transcranial Magnetic Stimulation in Participants With Vascular Depression,” The Journal of Neuropsychiatry and Clinical Neurosciences 22:75-84 (2010).
19. Normann et al., “Long-Term Plasticity of Visually Evoked Potentials in Humans is Altered in Major Depression,” Biological Psychiatry 62:373-380 (2007).
20. O'Reardon et al., “Efficacy and Safety of Transcranial Magnetic Stimulation in the Acute Treatment of Major Depression: A Multisite Randomized Controlled Trial,” Journal of Biological Psychiatry 62:1208-1216 (2007).
21. Paus et al., “Cortico-Cortical Connectivity of the Human Mid-Dorsolateral Frontal Cortex and its Modulation by Repetitive Transcranial Magnetic Stimulation,” European Journal of Neuroscience 14:1185-1415 (2001).
22. Perala, et al., “Lifetime Prevalence of Psychotic and Bipolar I Disorders in a General Population,” Arch Gen Psychiatry 64:19-28 (2007).
23. Pizzagalli, “Frontocingulate Dysfunction in Depression: Toward Biomarkers of Treatment Response,” Neuropsychopharmacology 36:183-206 (2011).
24. Pizzagalli et al., “Anterior Cingulate Activity as a Predictor of Degree of Treatment Response in Major Depression: Evidence From Brain Electrical Tomography Analysis,” The American Journal of Psychiatry 158:405-415 (2001).
25. Pogarell et al., “Striatal Dopamine Release After Prefrontal Repetitive Transcranial Magnetic Stimulation in Major Depression: Preliminary Results of a Dynamic [¹²³I] IBZM SPECT Study,” Journal of Psychiatric Research 40:307-314 (2006).
26. Reid et al., “Epidemiology of Chronic Non-Cancer Pain in Europe: Narrative Review of Prevalence, Pain Treatments and Pain Impact,” Current Medical Research and Opinion 27:449-462 (2011).
27. Sackeim et al., “Vagus Nerve Stimulation: A New Tool for Brain Research and Therapy,” Journal of Biological Psychiatry 47:287-295 (2000).
28. Shargorodsky et al., “Prevalence and Characteristics of Tinnitus Among US Adults,” The American Journal of Medicine 123:711-718 (2011).
29. Slotema et al., “Can Low-Frequency Repetitive Transcranial Magnetic Stimulation Really Relieve Medication-Resistant Auditory Verbal Hallucinations? Negative Results from a Large Randomized Controlled Trial,” Biological Psychiatry 69:450-456 (2010).
30. Strafella et al., “Striatal Dopamine Release Induced by Repetitive Transcranial Magnetic Stimulation of the Human Motor Cortex,” Brain 126:2609-2615 (2003).
31. Stubbeman et al., “Pretreatment Neurophysiologic Function and ECT Response in Depression,” The Journal of ECT 20(3):142-144 (2004).
32. Thirugnanasambandam et al., “Dose-Dependent Nonlinear Effect of L-DOPA on Paired Associative Stimulation-Induced Neuroplasticity in Humans,” The Journal of Neuroscience 31(4):5294-5299 (2010).
33. Thut et al., “New Insights into Rhythmic Brain Activity from TMS-EEG Studies,” Trends in Cognitive Sciences 182-189 (2009).
34. United States Patent Applications 20100003656, 20100004705, 20100004717, 20110282404.
35. Wand, “Cortical Changes in Chronic Low Back Pain: Current State of the Art and Implications for Clinical Practice,” Manual Therapy 16:15-20 (2011).

1. A therapeutic system comprising:
a non-invasive brain stimulation device configured to stimulate a patient's brain by emitting an electromagnetic field based on certain stimulation parameters;
a feedback device configured to measure data regarding brain activity;
a computer communicably connected to the feedback and stimulation devices, the computer configured to receive input from the feedback device and transmit an output to the brain stimulation device to adjust stimulation parameters.

2. The system according to claim 1 whereby the output comprises a signal designed to dynamically modify the brain stimulation device parameters to inhibit seizures if necessary.

3. The system according to claim 1 wherein the output comprises stimulation parameters designed to enhance neuroplasticity in the patient's brain.

4. The system according to claim 1 wherein the output comprises stimulation parameters designed to enhance neuroplasticity in the patient's brain in real-time.

5. The system according to claim 1 wherein the output comprises a signal designed to move the brain stimulation device to emit an electromagnetic field to a different part of the patient's brain.

6. The system according to claim 1 wherein the brain stimulation device is a transcranial magnetic stimulation device.

7. The system according to claim 1 wherein the feedback device is configured to perform quantitative electroencephalographic swLoreta brain imaging.

8. The system according to claim 1 further comprising a sensory stimulation device to provide sensory stimulation to the patient before, during, or after brain stimulation to treat a neurologic or psychiatric disorder or to enhance cognitive, motor, social, or psychological skills.

9. The system according to claim 8 wherein the sensory stimulation device is configured to deliver notched sound to treat tinnitus.

10. The system according to claim 8 wherein the sensory stimulation device is configured to deliver ambient noise reduction to treat auditory hallucinations.

11. The system according to claim 8 wherein the sensory stimulation device is configured to deliver uplifting music to treat depression.

12. The system according to claim 8 wherein the length or temporal relationship of a stimulus relative to another stimulus is designed to benefit treatment.

13. The system according to claim 1 wherein the brain stimulation device is a transcranial magnetic stimulation device, and wherein the system further comprises a sensory stimulation device configured to deliver notched white noise to treat tinnitus during brain stimulation.

14. The system according to claim 1 wherein the brain stimulation device is a transcranial magnetic stimulation device, and wherein the system further comprises a sensory stimulation device configured to deliver uplifting music for cognitive enhancement.

15. The system according to claim 1 wherein the brain stimulation device is a transcranial magnetic stimulation device, and wherein the system further comprises a sensory stimulation device configured to deliver relaxing music text to treat post-traumatic stress disorder.

16. The system according to claim 1 wherein the brain stimulation device is configured to emit burst stimulation.

17. A therapeutic system comprising:
a brain stimulation device configured to stimulate a patient's brain by emitting an electromagnetic field based on certain stimulation parameters;
a computer communicably connected to the brain stimulation device to transmit an output to the brain stimulation device with regard to stimulation parameters; and
a sensory stimulation device configured to provide sensory stimulation to the patient during brain stimulation to treat a neurologic or psychiatric disorder or to enhance cognitive, motor, social, or psychological skills.

18. The system according to claim 17 wherein the sensory stimulation device is configured to deliver notched white noise to treat tinnitus.

19. The system according to claim 17 wherein the sensory stimulation device is configured to deliver lyrics to treat auditory hallucinations.

20. The system according to claim 17 wherein the sensory stimulation device is configured to deliver uplifting music to treat depression.

21. The system according to claim 17 further comprising a feedback device configured to measure data regarding brain activity, and wherein the computer is configured to receive input from the feedback device and transmit an output to the brain stimulation device to adjust stimulation parameters.

22. The system according to claim 17 wherein the brain stimulation device is non-invasive.

23. The system according to claim 17 further comprising a second brain stimulation device configured to provide transcranial direct current stimulation, and wherein the first brain stimulation device is configured to provide transcranial magnetic stimulation.

24. The system according to claim 17 wherein the brain stimulation device is configured to emit burst stimulation.

25. The system according to claim 17 wherein the length or temporal relationship of a stimulus relative to another stimulus is designed to benefit treatment.

26. A method of therapeutic treatment, the method comprising:
providing electromagnetic stimulation to a brain; and
providing temporally associated sensory stimulation to treat a neurologic or psychiatric disorder or to enhance cognitive, motor, social, or psychological skills.

27. The method according to claim 26 wherein the sensory stimulation comprises notched white noise to treat tinnitus during brain stimulation.

28. The method according to claim 26 wherein the sensory stimulation comprises non-lyricized music to treat auditory hallucinations.

29. The method according to claim 26 wherein the sensory stimulation comprises uplifting music to treat depression.

30. The method according to claim 26 wherein the stimulation is burst stimulation.

31. The method according to claim 26 wherein the length or temporal relationship of a stimulus relative to another stimulus is designed to benefit treatment.

32. A therapeutic method comprising:
providing non-invasive electromagnetic stimulation to a brain using stimulation parameters;
measuring effects of brain stimulation; and
dynamically adjusting stimulation parameters to maximize treatment benefit.

33. The method according to claim 32 further comprising providing paired sensory stimulation to the patient before, during, or after brain stimulation to treat a neurologic or psychiatric disorder or to enhance cognitive, motor, social, or psychological skills.

34. A method using the system according to claim 1, the method comprising:
determining a primary brain stimulation strategy, secondary brain stimulation strategy, and sensory stimulation strategy;
applying the primary brain stimulation strategy to effect brain stimulation;
optionally applying the secondary brain stimulation strategy;
optionally applying the sensory stimulation strategy before, during, or after brain stimulation; and
optionally measuring effects of brain stimulation and adjusting stimulation parameters to maximize treatment benefit.

35. The method according to claim 34 wherein the primary brain stimulation strategy comprises delivering a transcranial magnetic stimulation, and wherein the sensory stimulation strategy comprises delivering notched white noise to treat tinnitus.

36. The method according to claim 34 wherein the primary brain stimulation strategy comprises delivering a transcranial magnetic stimulation, and wherein the sensory stimulation strategy comprises delivering uplifting music to treat depression.

37. The method according to claim 34 wherein the primary brain stimulation strategy comprises delivering a transcranial magnetic stimulation, and wherein the sensory stimulation strategy comprises delivering non-lyricized music to treat auditory hallucinations.

38. The method according to claim 34 wherein the length or temporal relationship of a stimulus relative to another stimulus is designed to benefit treatment.

39. A method of neuroplastic augmentation using brain stimulation designed to augment, hasten, enhance, optimize, or improve a secondary neurologic or psychiatric treatment for a brain illness, the method comprising:
determining a brain stimulation site using a feedback device;
determining brain stimulation parameters designed to augment neuroplasticity in the brain stimulation site to augment, enhance, optimize, or improve a secondary neurologic or psychiatric treatment for a brain illness;
providing electromagnetic stimulation to the brain stimulation site using the brain stimulation parameters before, during, or after the secondary neurologic or psychiatric treatment; and
measuring effects of brain stimulation using a feedback device.

40. The method according to claim 39 wherein the electromagnetic stimulation is burst stimulation.

41. A method of treating tinnitus comprising:
providing stimulation to the left dorsolateral prefrontal cortex, right dorsolateral prefrontal cortex, or cingulate cortex of a brain;
providing temporally associated sensory stimulation; and
providing stimulation to auditory cortical region of the brain.

42. The method according to claim 41 wherein the temporally associated sensory stimulation is notched sound.

43. The method according to claim 41 wherein the providing stimulation to auditory cortical region of the brain is inhibitory.

44. The method according to claim 43 wherein the inhibitory stimulation is theta burst stimulation.

45. The method according to claim 41 wherein the providing stimulation to the left dorsolateral prefrontal cortex, right dorsolateral prefrontal cortex, or cingulate cortex of a brain is electromagnetic stimulation.

46. The method according to claim 45 wherein the electromagnetic stimulation is burst stimulation.

47. The method according to claim 41 wherein the providing stimulation to auditory cortical region is stimulation to the right side if the tinnitus is equally loud on both sides.

48. The method according to claim 41 wherein the providing stimulation to auditory cortical region is stimulation provided contralateral to the side of loudest tinnitus.

49. The method according to claim 41 wherein the providing stimulation to auditory cortical region is stimulation provided to a side of the brain determined by measurement of brain activity.

50. The method according to claim 49 wherein the providing stimulation to auditory cortical region is stimulation provided to a side of the brain determined by measurement of auditory brain activity.

51. The method according to claim 50 wherein the providing stimulation to auditory cortical region is stimulation provided to a side of the brain determined by measurement of mismatch negativity.

52. The method according to claim 41 wherein the providing stimulation to the left dorsolateral prefrontal cortex, right dorsolateral prefrontal cortex, or cingulate cortex of a brain is ultrasonic stimulation.

53. The method according to claim 41 wherein the temporally associated sensory stimulation is brain stimulation directed at sensory cortical areas whereby peripheral sensory circuits are bypassed.

54. The method according to claim 41 wherein the length or temporal relationship of a stimulus relative to another stimulus is designed to benefit treatment.
